(19) United States
(12) Patent Application Publication
(10) Pub. No.: US 2007/0160577 A1
(43) Pub. Date: Jul. 12, 2007

(54) Interleukin-11 compositions and methods of use

(75) Inventors: Nitin K. Damle, Upper Saddle River, NJ (US); John DiJoseph, Woodbridge, NJ (US); Paul F. Schendel, Wayland, MA (US)

Correspondence Address: CHOATE, HALL & STEWART LLP, PATENT GROUP, TWO INTERNATIONAL PLACE, BOSTON, MA, 02110, US

(73) Assignee: Wyeth, Madison, NJ (US)

(21) Appl. No.: 11/634,502

(22) Filed: Dec. 6, 2006

Related U.S. Application Data
(60) Provisional application No. 60/742,658, filed on Dec. 6, 2005.
(60) Provisional application No. 60/842,294, filed on Sep. 5, 2006.

(51) Int. Cl. A61K 38/20; A61K 39/395

(52) U.S. Cl. 424/85.2; 424/144.1; 424/178.1

(57) ABSTRACT

The present invention provides methods for the treatment and/or prevention of thrombocytopenia including thrombocytopenia associated with drug-induced liver damage and thrombocytopenia associated with drug-induced bone marrow destruction. The methods of treatment of the invention include administration of interleukin-11 to a subject suffering from or susceptible to thrombocytopenia and/or receiving or about to receive a treatment involving a conjugate therapeutic agent whose administration results in thrombocytopenia. Also provided are pharmaceutical compositions and kits useful for carrying out such methods of treatment.

### RELATED APPLICATIONS

The present application claims priority from Provisional Application No. 60/742,658, filed Dec. 6, 2005 and Provisional Application No. 60/842,294 filed Sep. 5, 2006, both entitled “Interleukin-11 Compositions and Methods of Use”. Each of the provisional applications is incorporated herein by reference in its entirety.

### BACKGROUND OF THE INVENTION

Platelets are important for maintaining hemostasis and for initiating blood clot formation at sites of injury. Platelets also release growth factors at the site of clot formation that, among several functions, speed the healing process. In patients suffering from depressed levels of platelets (a condition known as thrombocytopenia), the inability to form clots is, the most immediate consequence. Severe thrombocytopenia results in a typical pattern of bleeding: multiple petechiae in the skin, often most evident on the lower legs; scattered small ecchymoses at sites of minor trauma; mucosal bleeding including nosebleed, gingival bleeding, bleeding in the gastrointestinal and genitourinary tracts; and excessive bleeding after surgery. Heavy gastrointestinal bleeding and bleeding of the central nervous system (CNS) may be life-threatening.

Thrombocytopenia manifests itself if either one of the steps in the thrombopoietic process is interfered with resulting in failed platelet production, abnormal platelet distribution, increased platelet destruction, and/or increased platelet consumption. The differentiation and proliferation of hematopoietic cells may be interfered with by either congenital or acquired causes, and these causes can vary widely. For example, congenital amegakaryocytic hypoplasia can selectively decrease production of megakaryocytes, the cells responsible for platelet production, thus resulting in thrombocytopenia. Low levels of circulating platelets may also occur after exposure to or treatment with a chemical agent or drug. Such drug-induced thrombocytopenia is generally treated by partial or complete withdrawal of the offending agent.

Thrombocytopenia can be a potentially fatal complication of many therapies for cancer including gamma irradiation, therapeutic exposure to radiation, cytotoxic chemotherapeutic drug treatment, and bone marrow transplantation. The diagnosis of thrombocytopenia in cancer patients is often complicated by the fact that such patients are often treated with multiple drugs and may also receive procedures that can enhance the toxicity of the drugs. Drug-induced thrombocytopenia can therefore limit the benefits of chemotherapy for potentially curable malignancies by preventing appropriate administration of drugs at the optimal doses and schedule, which can lead to an increase in cancer morbidity or even mortality.

### SUMMARY OF THE INVENTION

The present invention encompasses the recognition that certain chemotherapeutic agents, and in particular conjugates of protein targeting moieties with cytotoxic agents, pose particular risks with respect to development of thrombocytopenia. The invention provides a new system for the management of patients suffering from thrombocytopenia induced by administration of such agent. In particular, the present invention provides pharmaceutical compositions and methods that are useful for the prevention and/or treatment of thrombocytopenia, such as drug-induced thrombocytopenia (for example thrombocytopenia induced by chemotherapeutics, particularly conjugate chemotherapeutics). Inventive pharmaceutical compositions and methods of treatment may also be used for preventing or treating thrombocytopenia associated with liver damage (e.g., drug-induced liver damage). Alternatively or additionally, the inventive pharmaceutical compositions and methods of treatment may be used for preventing or treating thrombocytopenia associated with bone marrow destruction (e.g., drug-induced bone marrow destruction).

More specifically, in one aspect, the present invention provides methods of alleviating thrombocytopenia in a subject comprising a step of: administering a therapeutically effective amount of interleukin-11 to the subject suffering from or susceptible to thrombocytopenia, wherein thrombocytopenia is associated with administering to the subject a conjugate comprising a targeting moiety and a cytotoxic drug. Interleukin-11 used in the methods of the present invention may be, for example, recombinant human interleukin-11.

In certain embodiments of the methods of the present invention, the step of administering comprises administering a therapeutically effective amount of interleukin-11 to a subject suffering from cancer or a cancerous condition.

In certain embodiments, the targeting moiety in the conjugate whose administration results in thrombocytopenia, comprises an antibody, such as an anti-CD22 antibody, an anti-CD33 antibody, an anti-Lewis Y antibody, an anti-5T4 antibody, an anti-CD30 antibody, or any combinations thereof. The cytotoxic drug in the conjugate may be a calicheamicin, a calicheamicin derivative, an esperamicin, or an esperamicin derivative. For example, the conjugate may be an anti-CD22 antibody-calicheamicin conjugate, an anti-CD33 antibody-calicheamicin conjugate, an anti-Lewis Y antibody-calicheamicin conjugate, an anti-5T4 antibody-calicheamicin conjugate, or an anti-CD30 antibody-calicheamicin conjugate.

In certain embodiments of the methods of the present invention, interleukin-11 is administered prior to administration of the conjugate.

In certain embodiments, administration of interleukin-11 according to methods of the invention prevents, reduces, slows down or stops thrombocytopenia in the subject. Thrombocytopenia produced by administration of the conjugate may be, at least partly, resulting from bone marrow destruction. Alternatively or additionally, thrombocytopenia produced by administration of the conjugate may be, at least partly, resulting from liver damage. In such embodiments, administration of interleukin-11 may prevent, reduce, slow down or stop liver damage and/or liver damage-related inflammation in the subject.

In another aspect, the present invention provides pharmaceutical compositions comprising a therapeutically effective amount of interleukin-11, at least one conjugate whose administration results in thrombocytopenia, and at least one physiologically acceptable carrier, wherein the conjugate comprises a targeting moiety and a cytotoxic drug. In certain embodiments, interleukin-11 included in a pharmaceutical composition of the present invention, comprises recombinant human interleukin-11. In some embodiments, interleukin-11, at least one conjugate and at least one physiologically acceptable carrier are combined as one or more preparations for simultaneous or sequential administration of interleukin-11 and the conjugate.

The targeting moiety in the conjugate may be an antibody (e.g., an anti-CD22 antibody, an anti-CD33 antibody, an anti-Lewis Y antibody, an anti-5T4 antibody, an anti-CD30 antibody, or any combinations thereof), and the cytotoxic drug may be a calicheamicin, a calicheamicin derivative, an esperamicin, or an esperamicin derivative. For example, the conjugate included in an inventive pharmaceutical composition may be an anti-CD22 antibody-calicheamicin conjugate, an anti-CD33 antibody-calicheamicin conjugate, an anti-Lewis Y antibody-calicheamicin conjugate, an anti-5T4 antibody-calicheamicin conjugate, or an anti-CD30 antibody-calicheamicin conjugate.

In certain embodiments, administration of a pharmaceutical composition of the present invention to a subject prevents, reduces or stops thrombocytopenia in the subject. As mentioned above, the subject may suffer from cancer or a cancerous condition. Thrombocytopenia produced by administration of the conjugate may be, at least partly, resulting from bone marrow destruction. Alternatively or additionally, thrombocytopenia produced by administration of the conjugate may be, at least partly, resulting from liver damage. In such embodiments, administration of a pharmaceutical composition of the present invention may prevent, reduce, slow down or stop liver damage and/or liver damage-related inflammation in the subject.

In another aspect, the present invention provides a kit comprising: interleukin-11 and at least one conjugate whose administration results in thrombocytopenia. The conjugate comprises a targeting moiety (e.g., an antibody as described above) and a cytotoxic drug (e.g., a calicheamicin, a calicheamicin derivative, an esperamicin or an esperamicin derivative). Thrombocytopenia that results from administration of the conjugate may result, at least partly, from liver damage. Alternatively or additionally, thrombocytopenia that results from administration of the conjugate may result, at least partly, from bone marrow destruction.

These and other objects, advantages and features of the present invention will become apparent to those of ordinary skill in the art having read the following detailed description.

### BRIEF DESCRIPTION OF THE DRAWING

FIG. 1 is a graph showing the effects of IL-1 on CMC-544-induced thrombocytopenia in nude mice. The results presented on this figure were obtained as described in Example 1. Nude mice were administered vehicle alone, CMC-544 (200 μg/kg) alone, IL-11 alone, IL-11 (125 μg/kg sc) after administration of CMC-544 (200 μg/kg bid), or IL-11 (250 μg/kg sc) before and after administration of CMC-544 (200 μg/kg).

FIG. 2 is a graph showing the effects of IL-11 on CMC-544-induced thrombocytopenia in nude mice. The results presented on this figure were obtained as described in Example 1. Nude mice were administered vehicle alone, CMC-544 (160 μg/kg) alone, IL-11 (250 μg/kg sc) after administration of CMC-544 (160 μg/kg), or IL-11 (250 μg/kg sc) before and after administration of CMC-544 (160 μg/kg).

FIG. 3 is a graph showing the effects of intravenous administration of CMC-544 on the platelet counts of cynomolgus macaques. The results presented were obtained in an experiment where 9 monkeys were administered one dose of CMC-544 at 4.28 mg/m² (25 μg/kg calicheamicin) per week for four weeks (CMC-544 administration is designated by arrows in the graph). The platelet counts were followed as a function of time.

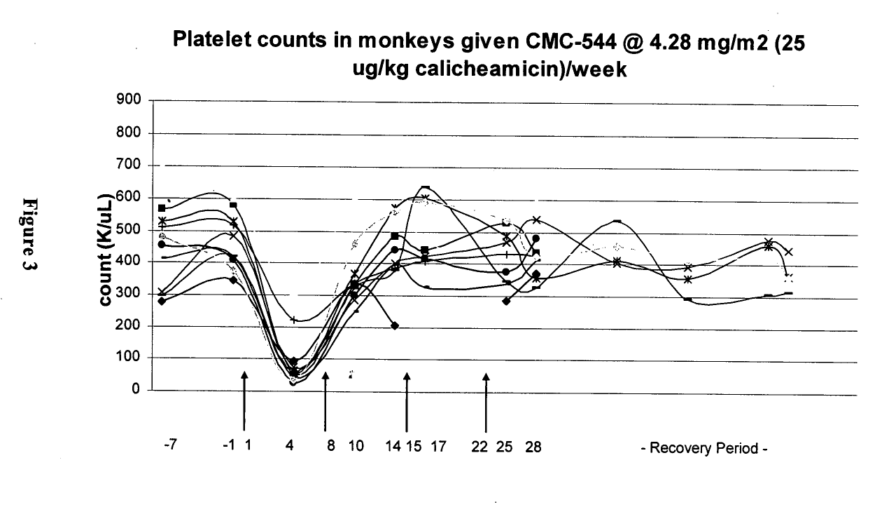

FIG. 4 is a graph showing the effects of IL-11 on CMC-544-induced thrombocytopenia in cynomolgus macaques. Concentrations of platelets are plotted as a function of the day of the procedure. Details of the experiments reported in this graph are described in Example 2. Four (4) test monkeys were administered IL-1 (represented by dark symbols in the graph) and four (4) control monkeys were administered the vehicle alone (represented by open symbols in the graph) following a similar administration schedule.

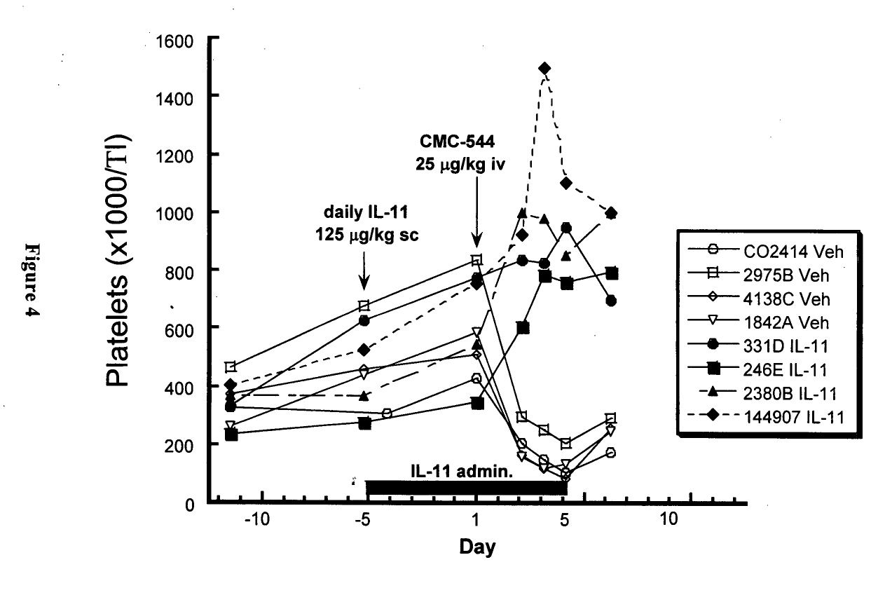

FIG. 5 presents two graphs showing the effects of IL-11 on CMC-544-induced increase in liver enzymes (ALT) in cynomolgus macaques. Details of the experiments are described in Example 2. The results presented on FIG. 5(A) were obtained for four (4) control monkeys that received CMC-544 and the vehicle alone. The results presented on FIG. 5(B) were obtained for four (4) test monkeys that received CMC-544 and IL-1.

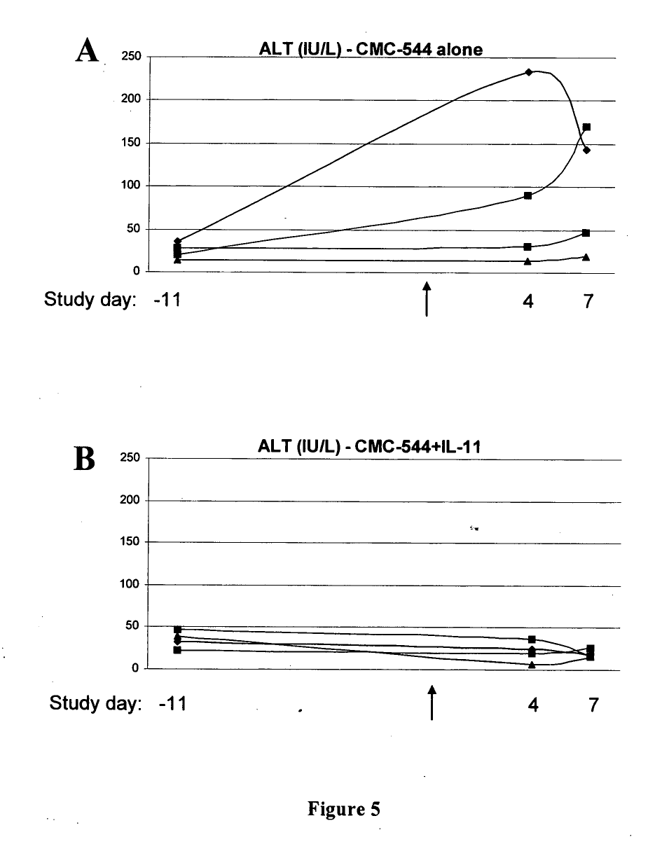

FIG. 6 presents two graphs showing the effects of IL-11 on CMC-544-related increased peripheral blood neutrophil counts in cynomolgus macaques. Details of the experiments are described in Example 2. The results presented on FIG. 6(A) were obtained for four (4) control monkeys that received CMC-544 and the vehicle alone. The results presented on FIG. 6(B) were obtained for four (4) test monkeys that received CMC-544 and IL-11.

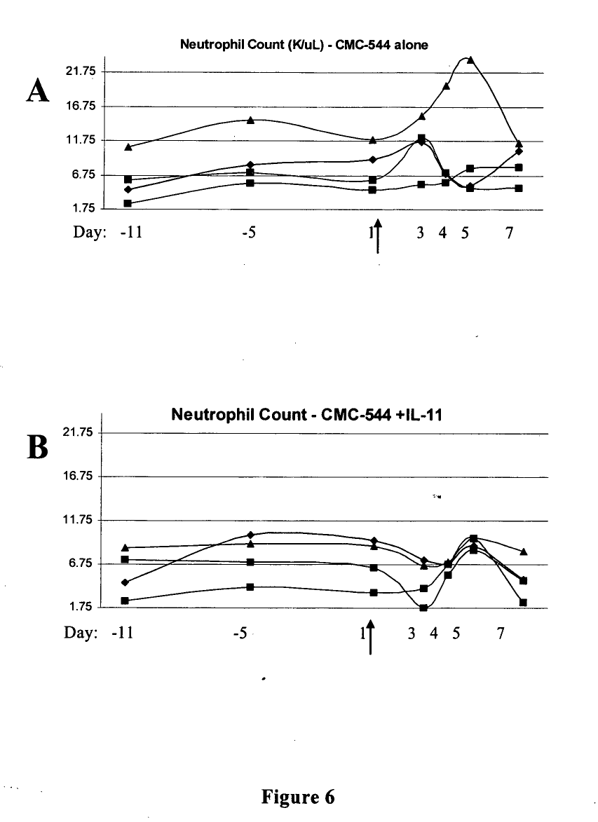

FIG. 7 presents two graphs showing the effects of IL-11 on CMC-544-related decreased serum albumin in cynomolgus macaques. Details of the experiments are described in Example 2. The results presented on FIG. 7(A) were obtained for four (4) control monkeys that received CMC-544 and the vehicle alone. The results presented on FIG. 7(B) were obtained for four (4) test monkeys that received CMC-544 and IL-11.

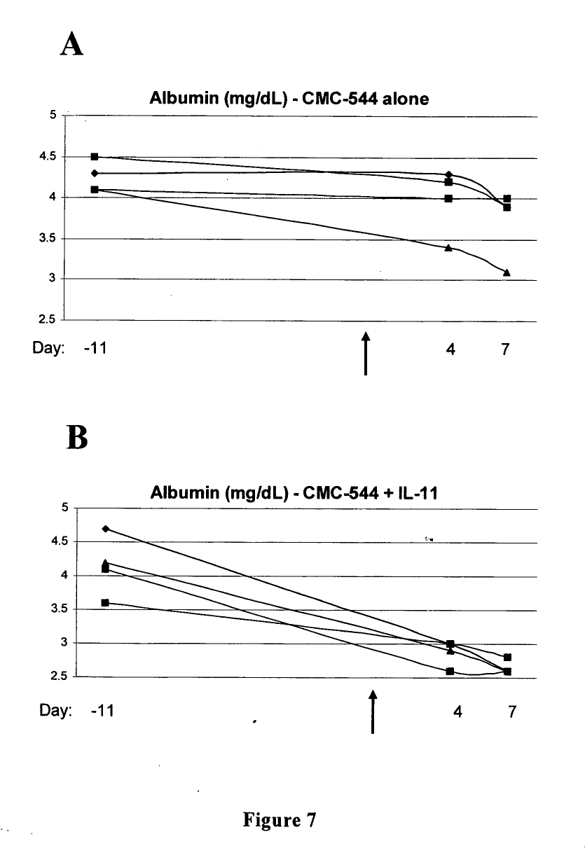

FIG. 8 presents two graphs showing the effects of IL-11 on CMC-544-related decrease in red cell mass (hemoglobin) in cynomolgus macaques. Details of the experiments are described in Example 2. The results presented on FIG. 8(A) were obtained for four (4) control monkeys that received CMC-544 and the vehicle alone. The results presented on FIG. 8(B) were obtained for four (4) test monkeys that received CMC-544 and IL-11.

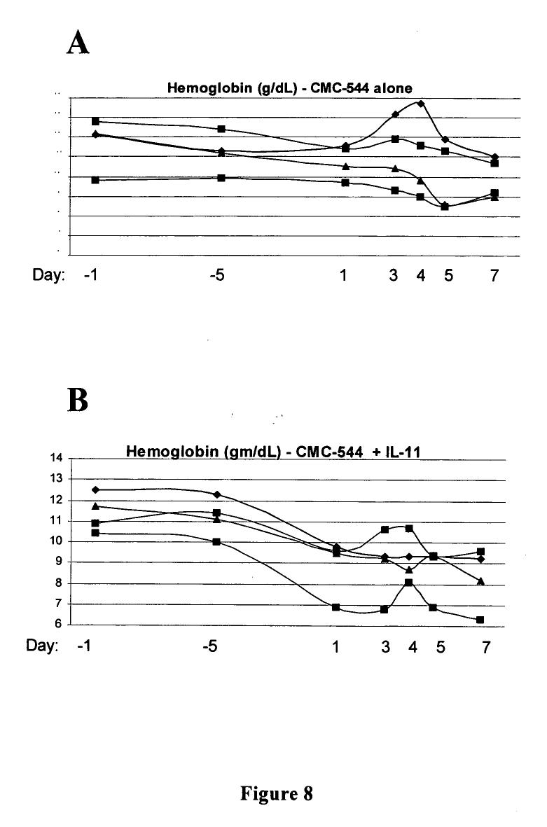

FIG. 9 presents two graphs showing the effects of administration of IL-11 in combination with CMC-544 on alkaline phosphatase in cynomolgus macaques. Details of the experiments are described in Example 2. The results presented on FIG. 9(A) were obtained for four (4) control monkeys that received CMC-544 and the vehicle alone. The results presented on FIG. 9(B) were obtained for four (4) test monkeys that received CMC-544 and IL-11.

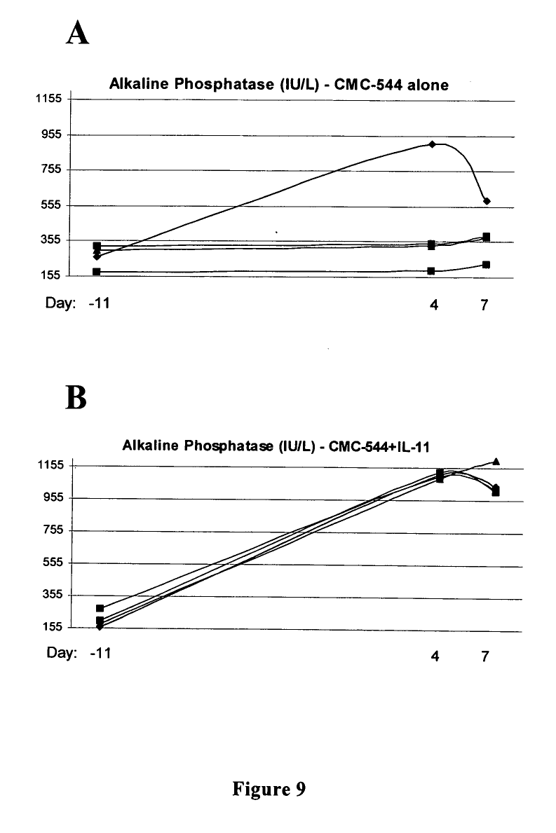

FIG. 10 presents two graphs showing the effects of administration of PBS (vehicle), CMC-544 or Carboplatin on circulating platelets (FIG. 10(A)) and circulating thrombopoietin (Tpo) (FIG. 10(B)) levels in nude mice. Details of the experiments are described in Example 5.

FIG. 11 presents a graph allowing comparison of the effects of administration of PBS (vehicle), CMC-544 or Carboplatin on circulating platelets levels and circulating thrombopoietin levels in nude mice. Details of the experiments are described in Example 5.

FIG. 12 presents two graphs showing the effects of administration of CMC-544 (FIG. 12(A)) and CMC-544+NEUMEGA (FIG. 12(B)) on platelet count (from pre-study to day 10) in cynomolgus macaques treated as described in the Second Study of Example 2.

FIG. 13 presents two graphs showing the effects of administration of CMC-544 (FIG. 13(A)) and CMC-544+NEUMEGA (FIG. 13(B)) on AST (from pre-study to day 14) in cynomolgus macaques treated as described in the Second Study of Example 2.

### DETAILED DESCRIPTION OF CERTAIN PREFERRED EMBODIMENTS

As mentioned above, the present invention encompasses the recognition that conjugate chemotherapeutics can pose particular risks for the development of thrombocytopenia in patients. Such conjugates generally include a protein-targeting moiety linked to a cytotoxic agent. According to the present invention, the protein-targeting moiety may target the conjugate chemotherapeutic for preferential metabolism in the liver, where the cytotoxic agent may induce damage that results in or exacerbates thrombocytopenia. Conjugates containing antibodies or other heavily glycosylated proteins may be particularly problematic in this regard.

The present invention encompasses the finding that administration of interleukin-11 can block drug-induced decrease in platelets circulating in the peripheral blood (i.e., thrombocytopenia). In addition, administration of IL-11 was found to block drug-induced liver damage, and to limit liver damage-related inflammation. Accordingly, the present invention provides pharmaceutical compositions and methods for the prevention and/or treatment of drug-induced thrombocytopenia, such as that resulting from liver damage and/or bone marrow destruction.

In the context of the present invention, a conjugate comprises a targeting moiety and a cytotoxic drug. Conjugates whose administration results in thrombocytopenia include any of a variety of conjugates of drugs known or suspected to cause, bring about, or stimulate the occurrence of thrombocytopenia or to be associated with one or more symptoms of thrombocytopenia. According to the present invention, administration of IL-11 prior to, following, or concomitant with administration of one or more of such conjugates alleviates thrombocytopenia. Specifically, such administration prevents, reduces, delays, treats or stops thrombocytopenia. As will be appreciated by one skilled in the art, administration of IL-11 may prevent, reduce or stop thrombocytopenia by preventing, reducing or stopping increased platelet destruction or consumption and/or by preventing, reducing or stopping decreased platelet production by the bone marrow.

I. Thrombocytopenia Resulting From Conjugate Administration

In the context of the present invention, thrombocytopenia, in a broad sense, refers to a physiological condition in mammals usually characterized by an abnormally low blood platelet count, typically resulting in easy bruising and abnormal bleeding from capillaries. In humans, the platelet count in the circulating blood is normally between 150 and 400 million per milliliter of blood (or 150 to 400×10⁹/L).

Platelets are produced in the bone marrow by large cells called megakaryocytes via a process called endomitosis (Y. Nagata et al., J. Cell Biol., 1997, 139: 449-457; L. Roy et al., Blood, 2001, 97: 2238-2247). In response to a decreased circulating platelet count, the rate of the endomitotic process increases, and the number of megakaryocytes in the bone marrow may increase up to 3-fold (L. A. Harker, J. Clin. Invest., 1968, 47: 458-465). This mechanism, in turn, causes the production and release into the circulation of additional platelets. In contrast, in response to an elevated circulating platelet count, the endomitotic rate decreases, and the number of megakaryocytes in the bone marrow may decrease by 50%. The exact physiological feedback mechanism by which the mass of circulating platelets regulates the endomitotic rate and number of bone marrow megakaryocytes is not fully understood. However, the major circulating factor involved in the feedback loop, and thus in platelet production, is thought to be thrombopoietin (K. Kaushansky et al., Proc. Natl. Acad. Sci. USA, 1995, 92: 3234-3238; K. Kaushansky, Thromb. Haemost., 1995, 74: 521-525), which is produced by hepatocytes in the liver (F. J. de Sauvage et al., Nature, 1994, 369; 533-538; R. Sungaran et al., Blood, 1997, 89: 101-107; S. Nomura et al., Exp. Hematol., 1997, 25: 565-572). Thus, in the context of the present invention, thrombocytopenia can result from bone marrow destruction, liver damage, or a combination of the two.

In some embodiments, administration of a conjugate results, at least in part, in liver damage, which ultimately results in thrombocytopenia. Conjugates whose administration results in liver damage generally include those conjugated cytotoxic agents whose administration causes, brings about or stimulates liver damage or is correlated with one or more symptoms of liver damage. In certain embodiments, such conjugates have a targeting moiety that is or includes a protein; in some embodiments the targeting moiety is or includes an antibody.

Liver damage according to the present invention includes not only degeneration or necrosis of liver parenchyma cells (hepatocytes) (e.g., which results from damage or injury caused by a certain factor), but also undesirable phenomena caused by biological reaction to the damage or injury, such as mobilization, infiltration, activation of Kupffer cells, leukocytes, and the like, swelling of the liver, fibrosis of the liver tissue, etc, which can occur alone or in combination. Liver injury, defect or dysfunction that is entirely or less than entirely caused, brought about or stimulated by administration of one or more conjugates is considered to be at least partly drug-induced. Thus, before administration of one or more conjugates, a subject may have a healthy liver (i.e., a liver showing no detectable sign of injury, defect, damage or dysfunction) or, alternatively, the subject may exhibit some existing level of liver injury/damage/defect/dysfunction (e.g., due to diseases, viruses, chemicals, drugs, or other factors).

In certain embodiments, administration of IL-11 prior to, following, or concomitant with administration of one or more conjugates prevents, reduces, delays, treats or stops thrombocytopenia resulting, at least in part, from conjugate-induced liver damage.

In some embodiments, administration of a conjugate results, alternatively (or in some cases additionally), at least in part, in bone marrow destruction, which produces thrombocytopenia. Conjugates whose administration results in bone marrow destruction include those conjugated cytotoxic agents whose administration causes, brings about, stimulates bone marrow destruction or is correlated with one or more symptoms of bone marrow destruction. Bone marrow destruction (i.e., bone marrow damage, defect or dysfunction) includes any condition affecting the bone marrow that results in abnormally low platelet production.

According to the present invention, administration of IL-11 prior to, following, or concomitant with administration of one or more conjugates prevents, reduces, delays, treats or stops thrombocytopenia resulting, at least partly, from bone marrow destruction. In certain embodiments, administration of IL-11 according to the present invention prevents, reduces or stops decreased production of platelets due to bone marrow destruction. Before administration of the conjugate inducing bone marrow destruction, a subject may have a healthy bone marrow (i.e., a bone marrow showing no detectable sign of destruction, damage, defect or dysfunction) or alternatively, the subject may exhibit some existing level of bone marrow destruction/defect/dysfunction (e.g., due to cancer, such as leukemia or lymphoma; viral infection or aplastic anemia or caused by toxic chemicals, radiation therapy or previous chemotherapy).

II. Conjugates

A conjugate generally is a molecule resulting from the bonding of at least two other molecules. The bonding between the two molecules may be covalent or non-covalent. As already mentioned above, a conjugate herein comprises a targeting moiety and a cytotoxic drug.

Targeting moieties are entities that have some degree of attraction for a target of interest when comprised in a conjugate. A targeting moiety often exhibits high affinity and/or specificity for the target, i.e., it specifically and/or efficiently recognizes, interacts with, binds to, or labels the target under the conditions or circumstances of its exposure to the target. A target may be a specific tissue or organ in the body, a specific type of cells or a specific cell component (e.g., cell surface receptor or antigen). Targeting moieties may desirably be stable, non-toxic entities that retain their properties under in vitro and/or in vivo conditions. The interaction between a targeting moiety and a target may be covalent or non-covalent. Most often, the interaction between a targeting moiety and a target is non-covalent. Examples of non-covalent interactions include, but are not limited to, hydrophobic interactions, electrostatic interactions, dipole interactions, van der Waals interactions, and hydrogen bonding. Irrespective of the nature of the interaction, the binding between a target and a targeting moiety within a conjugate is preferably selective, specific, and strong enough to allow the drug to play its role (e.g., exert its anti-cancer activity if the cytotoxic drug is a chemotherapeutics).

Within a conjugate, a cytotoxic drug may be associated with the targeting moiety in any of a variety of ways. In many embodiments, the drug is covalently attached to the targeting moiety. As will be appreciated by those skilled in the art, the drug and targeting moiety may be attached to each other either directly or indirectly (e.g., through a linker).

In certain embodiments, the cytotoxic drug and targeting moiety are directly, covalently linked to each other. The direct covalent binding can be through a linkage such as an amide, ester, carbon-carbon, disulfide, carbamate, ether, thioether, urea, amine, or carbonate linkage. The covalent binding can be achieved by taking advantage of functional groups present on the drug and the targeting moiety. Suitable functional groups that can be used to attach the two moieties together include, but are not limited to, amines, anhydrides, hydroxy groups, carboxy groups, and thiols. An activating agent, such as a carbodiimide, can be used to form a direct linkage. A wide range of activating agents are known in the art and are suitable for linking a drug and a targeting moiety.

In other embodiments, the cytotoxic drug and targeting moiety are indirectly covalently linked to each other via a linker group. This can be accomplished by using any number of stable bifunctional agents well known in the art, including homofunctional and heterofunctional linkers (see, for example, Pierce Catalog and Handbook). The use of a bifunctional linker differs from the use of an activating agent in that the former results in a linking moiety being present in the resulting conjugate, whereas the latter results in a direct coupling between the two moieties involved in the reaction. The role of the bifunctional linker may be to allow the reaction between two otherwise inert moieties. Alternatively or additionally, the bifunctional linker, which becomes part of the reaction product, may be selected such that it confers some degree of conformational flexibility to the conjugate. Alternatively or additionally, the bifunctional linker may be selected such that the linkage formed between the drug and the targeting moiety is hydrolysable (for examples of such linkers, see e.g. U.S. Pat. Nos. 5,773,001; 5,739,116 and 5,877,296). Such linkers are preferably used when higher activity of the drug is observed after hydrolysis of the targeting moiety. Exemplary mechanisms by which a drug is cleaved from the targeting moiety (e.g., antibody) include hydrolysis in the acidic pH of the lysosomes (hydrazones, acetals, and cis-aconitate-like amides), peptide cleavage by lysosomal enzymes (the capthepsins and other lysosomal enzymes), and reduction of disulfides.

One example of a suitable conjugate relies on the conjugation of hydrazides and other nucleophiles to the aldehydes generated by oxidation of the carbohydrates that naturally occur on antibodies. Hydrazone-containing conjugates can be made with introduced carbonyl groups that provide the desired drug-release properties. Conjugates can also be made with a linker that has a disulfide at one end, an alkyl chain in the middle, and a hydrazine derivative at the other end. The anthracyclines are one example of cytotoxins that can be conjugated to antibodies using this technology.

Linkers containing functional groups other than hydrazones have the potential to be cleaved in the acidic milieu of the lysosomes. For example, conjugates can be made from thiol-reactive linkers that contain a site other than a hydrazone that is cleavable intracellularly, such as esters, amides, and acetals/ketals. Camptothecin is one cytotoxic agent that can be conjugated using these linkers. Ketals made from a 5 to 7-member ring ketone and that has one of the oxygen atoms attached to the cytotoxic agent and the other to a linker for antibody attachment also can be used. The anthracyclines are again an example of a suitable cytotoxic agent for use with these linkers.

Another example of class of pH sensitive linkers are the cis-aconitates, which have a carboxylic acid group juxtaposed to an amide group. The carboxylic acid accelerates amide hydrolysis in the acidic lysosomes. Linkers that achieve a similar type of hydrolysis rate acceleration with several other types of structures can also be used. The maytansinoids are an example of cytotoxin that can be conjugated with linkers attached at C-9.

Another potential release method for drug conjugates is the enzymatic hydrolysis of peptides by the lysosomal enzymes. In one example, a peptide is attached via an amide bond to para-aminobenzyl alcohol and then a carbamate or carbonate is made between the benzyl alcohol and the cytotoxic agent. Cleavage of the peptide leads to the collapse, or self-immolation, of the aminobenzyl carbamate or carbonate. The cytotoxic agents exemplified with this strategy include anthracyclines, taxanes, mitomycin C, and the auristatins. In one example, a phenol can also be released by collapse of the linker instead of the carbamate. In another variation, disulfide reduction is used to initiate the collapse of a para-mercaptobenzyl carbamate or carbonate.

Many cytotoxic agents have little, if any, solubility in water and that can limit drug loading on the conjugate due to aggregation of the conjugate. One approach to overcoming this is to add solubilizing groups to the linker. Conjugates made with a linker consisting of PEG and a dipeptide can be used, including those having a PEG di-acid, thiol-acid, or maleimide-acid attached to the targeting moiety (e.g., antibody), a dipeptide spacer, and an amide bond to the amine of an anthracycline or a duocarmycin analogue. Another example is conjugates that are made with a PEG-containing linker disulfide bonded to a cytotoxic agent and amide bonded to an antibody. Approaches that incorporate PEG groups may be beneficial in overcoming aggregation and limits in drug loading.

A. Targeting Antibodies

In certain embodiments, the conjugate comprises an antibody as targeting moiety. These types of conjugates are commonly referred to as immunoconjugates, with those conjugates having a radioisotope as the drug, referred to as radioimmunoconjugates and those having a chemotherapeutic agent as the drug, referred to as chemoimmunoconjugates. Generally speaking, an antibody for these purposes may be any immunoglobulin (i.e., an intact immunoglobulin molecule, an active portion of an immunoglobulin molecule, etc), that binds to a specific epitope. The term encompasses monoclonal antibodies and antibody compositions with polyepitopic specificity (i.e., polyclonal antibodies).

Targeting antibodies can be from almost any mammalian species (e.g., mouse, human, primate, dog, etc) and can be produced by various methods well known in the art (e.g., murine antibodies via hybridomas, human antibodies via hybridomas from transgenic mice, etc).

Examples of antibodies that may be used in the formation of conjugates useful in the present invention include monoclonal antibodies (mAbs), for example, chimeric antibodies, humanized antibodies, primatized antibodies, resurfaced antibodies, human antibodies and biologically active fragments thereof. As already mentioned above, the term antibody is used broadly to refer to both antibody molecules and a variety of antibody derived molecules. Such antibody-derived molecules generally comprise at least one complementarity determining region (CDR) from either a heavy chain or light chain variable region, including molecules such as Fab fragments, F(ab′)₂ fragments, Fd fragments, Fabc fragments, Sc antibodies (single chain antibodies), diabodies, individual antibody light single chains, individual antibody heavy chains, chimeric fusions between antibody chains and other molecules, and the like. Antibodies mimetics having binding affinity for an antigen but not having one or more traditional CDRs also can be used in the formation of conjugates useful in the present invention.

In certain embodiments, a targeting antibody of a conjugate is directed against one or more cell surface antigens expressed on target cells and/or tissues in proliferative disorders such as cancer.

Examples of specific antibodies directed against cell surface antigens on target cells include without limitation, antibodies against CD22 antigen, which is over-expressed on most B-cell lymphomas; G5/44, a humanized form of a murine anti-CD22 monoclonal antibody; antibodies against cell surface antigen CD33, which is prevalent on certain human myeloid tumors especially acute myeloid leukemia (see, for example U.S. Pat. Appln. Nos. 2004-0192900 and 2004-0082764, each of which is incorporated herein by reference in its entirety); hP67.6, a humanized form of the anti-CD33 murine antibody (see, U.S. Pat. No. 5,773,001, which is incorporated herein by reference in its entirety); an antibody against PEM antigen found on many tumors of epithelial origin designated mP67.6 (see, for example, I. D. Bernstein et al., J. Clin. Invest., 1987, 79: 1153-1159 and I. D. Bernstein et al., J. Immunol., 1992, 128: 867-881, each of which is incorporated herein by reference in its entirety), and a humanized antibody against the Lewis Y carbohydrate antigen overexpressed on many solid tumors and designated hu3S193 (see, for example, U.S. Pat. No. 6,310,185, which is incorporated herein by reference in its entirety).

Other suitable antibodies include antibodies directed against the 5T4 oncofetal antigen. The 5T4 antigen is a 72 kDa highly glycosylated transmembrane glycoprotein comprising a 42 kDa non-glycosylated core (Hole et al., Br. J. Cancer, 1988, 57: 239-246; Hole et al., Int. J. Cancer, 1990, 45: 179-184; WO 89/07947; U.S. Pat. No. 5,869,053, each of which is incorporated herein by reference in its entirety). 5T4 includes an extracellular domain characterized by two leucine-rich repeats (LRRs) and an intervening hydrophilic region, which is an accessible antigen for targeted therapy (Myers et al., J. Biol. Chem., 1994, 269: 9319-9324). Other suitable antibodies include antibodies directed against CD30 antigen, which is over-expressed on a variety of hematologic malignancies. CD30 is an attractive target for cancer therapy because it has minimal expression on normal tissues. SGN-30 is an example of an anti-CD30 antibody that has been shown to induce direct anti-cancer activity towards tumor cells expressing CD30 (A. Forero et al., J. Clin. Oncol., 2005: Vol. 23, No. 16S: 6601).

In addition, there are several commercially available antibodies directed against cell surface antigens, such as rituximab (Rituxan™) and trastuzumab (Herceptin™), which may be used as targeting moieties in chemotherapeutic conjugates. Rituximab (Rituxan™) is a chimeric anti-CD20 antibody used to treat various B-cell lymphomas and trastuzumab (Herceptin™) is a humanized anti-Her2 antibody used to treat breast cancer.

In certain embodiments of the invention, a targeting moiety of a chemotherapeutic conjugate is an anti-CD22 antibody, an anti-CD33 antibody, an anti-Lewis Y antibody, an anti-CD30 antibody, or an anti-5T4 antibody.

B. Cytotoxic Drugs

Suitable cytotoxic drugs include any of a large variety of substances, molecules, compounds, agents, or factors that are toxic to living cells. Administered as a conjugate to a patient, a suitable cytotoxic drug is associated with thrombocytopenia. Thrombocytopenia may result, at least partly, from drug-induced liver damage. Alternatively or additionally, thrombocytopenia may result at least partly, from drug-induced bone marrow destruction.

As will be appreciated by one of ordinary skill in the art, a cytotoxic drug may be a synthetic or a natural compound; a single molecule or a complex of different molecules. Suitable cytotoxic drugs can belong to any of various classes of compounds including, but not limited to, small molecules, peptides, saccharides, steroids, antibodies, fusion proteins, antisense polynucleotides, ribozymes, small interfering RNAs, peptidomimetics, and the like. When cytotoxic drugs are used in the treatment of cancer or a cancerous condition, they can be found among the following classes of anti-cancer drugs: alkylating agents, anti-metabolite drugs, anti-mitotic antibiotics, alkaloidal anti-tumor agents, hormones and anti-hormones, interferons, non-steroidal anti-inflammatory drugs, and various other anti-tumor agents.

Examples of suitable drugs for use in immunoconjugates include the taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers such as those employed in photodynamic therapy, can also be used in immunoconjugates. In addition, conjugates can be made using secondary carriers as the cytotoxic agent, such as liposomes or polymers, for example.

In certain embodiments, the cytotoxic drug belongs to the enediyne family of antibiotics. As a family, the enediyne antibiotics are the most potent, anti-tumor agents discovered so far. Some members are 1000 times more potent than adriamycin, one of the most effective, clinically used anti-tumor antibiotics (Y. S. Zhen et al., J. Antibiot., 1989, 42: 1294-1298).

In certain embodiments, the cytotoxic drug is a member of the enediyne family of calicheamicins. Originally isolated from a broth extract of the soil microorganism Micromonospora echinospora ssp. calichensis, the calicheamicins were detected in a screen for potent DNA damaging agents (M. D. Lee et al., J. Am. Chem. Soc., 1987, 109: 3464-3466; M. D. Lee et al., J. Am. Chem. Soc., 1987, 109: 3466-3468; W. M. Maiese et al., J. Antibiot., 1989, 42: 558-563; M. D. Lee et al., J. Antibiot., 1989, 42: 1070-1087).

Calicheamicins are characterized by a complex, rigid bicyclic enediyne allylic trisulfide core structure linked through glycosyl bonds to an oligosaccharide chain. The oligosaccharide portion contains a number of substituted sugar derivatives, and a substituted tetrahydropyran ring. The enediyne containing core (or aglycone) and carbohydrate portions of calicheamicins have been reported to carry out different roles in the biological activity of these molecules. Without wishing to be bound by any particular theory, we note that it is generally believed that the core portion cleaves DNA, whereas the oligosaccharide portion of the calicheamicins serves as a recognition and delivery system and guides the drug to a double-stranded DNA minor groove in which the drug anchors itself. Once positioned in the minor groove of DNA, the enediyne core undergoes an electronic rearrangement (Bergman cyclization) to form a transient 1,4-benzenoid diradical. Formation of the diradical intermediate can be triggered by the presence of reducing agents such as NADPH or dithiothrietol. The diradical species provides the thermodynamic driving force for the DNA cleaving reaction by promoting hydrogen atom abstraction from the deoxyriboses. Reaction of the resulting deoxyribose carbon-centered radicals with molecular oxygen initiates a process that results in both single-strand and double-strand DNA cleavages (“Enediyne Antibiotics as Antitumor Agents”, Doyle and Borders, 1995, Marcel-Dekker: New York; N. Zein et al., Science, 1988, 240: 1198-1201; N. Ikemoto et al., Proc. Natl. Acad. Sci., USA, 1995, 92: 10506-10510; A. G. Myers et al., J. Am. Chem. Soc., 1994, 116: 1255-1271: M. D. Lee et al., Acc. Chem. Res., 1991, 24: 235-243; Y. Xu et al., Biochemistry, 1997, 36: 14975-14984). Double-stranded DNA cleavage is a type of damage that is usually non-repairable or non-easily repairable for the cell and is most often lethal.

Because of their chemical and biological properties, several analogues of the calicheamicins have been tested in preclinical models as potential anti-tumor agents. Their development as single agent therapies has not been pursued because of delayed toxicities that limit the therapeutic dose range for treatment. However, their potency makes them particularly useful for conjugate-targeted chemotherapy, where limits in expression of the conjugate target can require high potency in order to achieve effectiveness (C. Liu and R. V. J. Chri, Exp. Opin. Invest. Drugs, 1997, 6: 169-172; R. V. J. Chari et al., Cancer Res., 1995, 55: 4079-4084).

Accordingly, in certain embodiments of the present invention, the chemotherapeutic comprises an antibody-calicheamicin conjugate.

It will be appreciated that the term “calicheamicin” can refer to any member of the family of antibacterial and anti-tumor agents known as calicheamicins, for example, as described in U.S. Pat. Nos. 4,970,198 and 5,108,912 (each of which is incorporated herein by reference in its entirety). Analogs or derivatives of calicheamicins, such as, for example, N-acyl derivatives described in U.S. Pat. No. 5,079,233 (which is incorporated herein by reference in its entirety); disulfide analogs of calicheamicin (e.g., as described in U.S. Pat. Nos. 5,606,040; and 5,770,710, each of which is incorporated herein by reference in its entirety); dihydro derivatives (e.g., as described in U.S. Pat. No. 5,037,651, which is incorporated herein by reference in its entirety); and N-acetylated derivatives (e.g., as described in U.S. Pat. No. 5,079,233, which is incorporated herein by reference in its entirety) can be employed.

Several antibody-calicheamicin conjugates have been prepared and tested for their anti-tumor properties (L. M. Hinmam et al., Cancer Res., 1993, 53: 3336-3342; P. R. Hamann et al., Bioconj. Chem., 2005, 16: 346-353; N. K. Damle and P. Frost, Curr. Opin. Pharmacol., 2003, 3: 386-390). In certain embodiments, an antibody-calicheamicin conjugate can be included in an inventive pharmaceutical composition or used in an inventive method of treatment; such conjugates include those conjugates described in U.S. Pat. Nos. 5,773,001; 5,739,116; 5,712,374; 5,714,586; and 5,877,296; PCT application WO 03/092623, each of which is incorporated herein by reference in its entirety.

In certain embodiments of the present invention, the antibody-calicheamicin conjugate is CMC-544. CMC-544 is targeted to CD22 expressed by B-lymphoid malignancies. CMC-544 comprises a humanized IgG4 anti-CD22 monoclonal antibody (mAb), G5/44, covalently linked to N-acetyl-γ-calicheamicin dimethyl hydrazide (CalichDMH) via an acid-labile 4-(4′-acetylphenoxy) butanoic acid linker. CMC-544 can be prepared, for example, as described in J. F. DiJoseph et al., Blood, 2004, 103: 1807-1814 and U.S. Pat. Appln. Nos. 2004-0082764A1 and 2004-0192900A1, each of which is incorporated herein by reference in its entirety.

In other embodiments, the antibody-calicheamicin conjugate is CMD-193, which is described in U.S. patent application Ser. No. 10/080,587 (incorporated herein by reference in its entirety). CMD-193 is N-acetyl-γ-calicheamicin dimethyl hydrazide covalently attached to the anti-Lewis Y antibody G193 with the average loading of calicheamicin conjugate from about 5 to about 7 moles of calicheamicin per mole of antibody and the low conjugated fraction (LCF) of the conjugate less than about 10%.

In other embodiments, the antibody-calicheamicin conjugate is MYLOTARG®, also known as, CMA-676, CMA, or gemtuzumab ozogamicin (see, for example, E. L. Sievers et al., Blood, 1999, Blood, 93: 3678-3584, and U.S. Pat. Nos. 5,712,374; 5,714,586; 5,739,116; 5,767,285; 5,773,001; 5,877,296 and U.S. Pat. Application No. 2004-0152632, each of which is incorporated herein by reference). MYLOTARG® is currently approved for the treatment of acute myeloid leukemia in elderly patients. The conjugate consists of an antibody against CD33 that is bound to calicheamicin by means of an acid-hydrolysable linker. The disulfide analog of the semi-synthetic N-acetyl-γ-calicheamicin is used in the conjugation (U.S. Pat. Nos. 5,606,040 and 5,770,710).

In other embodiments, the antibody-calicheamicin conjugate is CME-548 (see, for example, U.S. patent application Ser. No. 11/221,902 (incorporated herein by reference in its entirety)).

In certain other embodiments, the cytotoxic drug belongs to the enediyne family of esperamicins. Esperamicins have been identified in cultures of Actinomadura verrucosospora (M. Konishi et al., J. Antibiot., 1985, 38: 1605-1609), and the elucidation of their structures has been reported (J. Golik et al., J. Am. Chem. Soc., 1987, 109: 3461-3462; J. Golik et al., J. Am. Chem. Soc., 1987, 109: 3462-3464). The mechanism by which these molecules produce cytotoxicity was investigated and found to be similar to that of calicheamicins and involve the participation of a diradial species which leads to the formation of single- and double-stranded DNA breaks (B. H. Long et al., Proc. Natl. Acad. Sci. USA, 1989, 86: 2-6).

In certain embodiments of the present invention, the chemotherapeutic conjugate is an antibody-esperamicin conjugate. As will be appreciated, the term “esperamicin” can refer to any member of the esperamicin family of antibacterial and anti-tumor agents known in the art; analogs or derivatives of such esperamicins may also be employed (see, for example, U.S. Pat. Nos. 4,675,187; 4,539,203; 4,554,162; and 4,837,206, each of which is incorporated herein by reference in its entirety).

III. Interleukin-11

The present invention provides methods of administration of, and pharmaceutical compositions comprising, interleukin-11 (IL-11).

Interleukin-11 is a member of a family of growth factors that includes growth hormone, granulocyte colony-stimulating factor (G-CSF), and other growth factors. IL-11 is also a member of a family of cytokines that includes IL-6, leukemia inhibitory factor (LIF), oncostatin M (OSM), and ciliary neurotrophic factor (CNTF), which all signal through a common receptor subunit, gp130 (S. Neben and K. Turner, Stem Cells, 1993, 11: 156-162). IL-11, which is naturally produced by bone marrow stromal cells, is a thrombopoietic growth factor that, in conjunction with other factors, stimulates the proliferation of hematopoietic stem cells and megakaryocyte progenitor cells and induces maturation resulting in increased platelet production.

Typically, inventive methods and compositions utilize IL-11 in an active form, and often in an active form substantially free from association with other mammalian proteins or proteinaceous materials.

Interleukin-11 (or IL-11), as used in accordance with the present invention, is generally an isolated protein comprising the entire polypeptide sequence of wild-type or mutant IL-11 or an active fragment thereof. A protein or polypeptide may be considered isolated by virtue of its origin or manipulation, for example if (a) it is present in a host cell as the expression product of a portion of an expression vector; or (b) it is linked to a protein or chemical moiety other than that to which it is linked in nature; or (c) it does not occur in nature. Alternatively or additionally, an isolated polypeptide or protein may be one that is produced or prepared (including by chemical synthesis), by the hand of man. Those of ordinary skill in the art will appreciate that a wild-type polypeptide or protein has a normal amino acid sequence found in nature, whereas a mutant polypeptide or protein has an amino acid sequence that is identical to that of the wild type at most positions but that includes one or more differences (e.g., substitutions, additions, deletions, alterations or combinations thereof) at precise locations. A mutant can have more than one difference but, as can be appreciated by those of ordinary skill in the art, overall sequence similarity to the wild-type is maintained.

In certain embodiments, IL-11 utilized in accordance with the present invention has the sequence of naturally occurring human IL-11 or of other mammals (e.g., mouse, rat, rabbit, monkey, dog, cat, pig, cow, horse and the like). The molecular cloning and characterization of murine interleukin-11 has been reported by J. C. Norris et al., (Exp. Hematol., 1996, 24: 1369-1376, which is incorporated herein by reference in its entirety). The cDNA sequence and the amino acid sequence (single letter code) of primate (healthy macaque monkey) and human clones of the IL-11 polypeptide can be found in U.S. Pat. Nos. 5,371,193; 5,700,664; 5,854,028 and 6,066,317 (each of which is incorporated herein by reference in its entirety). The primate nucleotide sequence comprises 1100 base pairs, including a 5′ non-coding sequence of 72 bases and a 3′ non-coding sequence of 431 bases. The human nucleotide sequence similarly contains a single long reading frame of 597 nucleotides. Both the primate and human IL-11 proteins have a molecular mass of approximately 19,000 daltons; are 178 amino acids in length; and are non-glycosylated. A polynucleotide that encodes primate or human IL-11 has been disclosed in U.S. Pat. No. 5,215,895 (which is incorporated herein by reference in its entirety).

Thus, in certain embodiments, IL-11 included in a pharmaceutical composition or used in a method of treatment of the present invention comprises murine IL-11. In other embodiments, the IL-11 comprises primate IL-11. In still other embodiments, IL-11 comprises human IL-11. In some embodiments, the type of IL-11 protein utilized matches that of the target species (i.e., species of subjects to undergo treatment with IL-11). For instance, in some embodiments, human IL-11 is used in compositions and methods for treatment of humans.

In other embodiments, the amino acid sequence of IL-11 to be used in the compositions and methods of the present invention is sufficiently homologous to naturally occurring IL-11 (e.g., to one or more sequences published in references cited above and/or listed in an established database such as GenBank, SwissProt, etc). Typically, polypeptides or proteins are considered sufficiently homologous to naturally occurring IL-11 if they share overall sequence identity of at least 35% with the IL-11. In certain embodiments, the sequence identity is at least 40%, 60%, 80%, 90%, 95%, 96%, 97%, 98%, 99% or better. Calculations of the percent homology or identity of two amino acid sequences can be performed by aligning the two sequences for optimal comparison purposes (e.g., gaps can be introduced in one or both of a first and a second amino acid sequence for optimal alignment and non-homologous sequences can be disregarded for comparison purposes). In certain embodiments, the length of a reference sequence aligned for comparison purposes is at least 30%, 40%, 60%, 80%, 90%, 95% or more, e.g., 96%, 97%, 98%, 99% or 100% of the length of the reference sequence. The amino acid residues at corresponding amino acid positions are then compared. When a position in the first sequence is occupied by the same amino acid residue as the corresponding position in the second sequence, then the molecules are identical (or homologous) at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences may be determined using the Needleman and Wunsch algorithm (J. Mol. Biol., 1970, 48: 444-453), which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. The percent identity between two amino acid sequences can also be determined using the algorithm of Meyers and Miller (CABIOS, 1989, 4: 11-17), which has been incorporated into the ALIGN program (version 2.0), using a PAM120 weight residue table, a gap length penalty of 12 and a gap penalty of 4.

Compared to the amino acid sequence of naturally occurring IL-11, a sufficiently homologous sequence may include one or more of conservative substitutions, additions, alterations or deletions of one or more selected amino acid residues. As will be understood by those of ordinary skill in the art, conservative substitutions generally represent substitutions that are physically or functionally similar to the corresponding reference residue, e.g., that have a similar size, shape, electric charge, chemical properties, including the ability to form covalent or hydrogen bonds, or the like. In some embodiments, conservative substitutions are those fulfilling the criteria defined for an “accepted point mutation” by Dayhoff et al. (“Atlas of Protein Sequence and Structure”, 1978, Nat. Biomed. Res. Foundation, Washington, D.C., Suppl. 3, 22: 354-352). Techniques for such replacement, insertion or deletions of individual residues or sets of residues are well known in the art (see, for example, U.S. Pat. No. 4,518,584).

In some embodiments of the invention, the amino acid sequence of IL-11 to be used in the compositions and methods of the present invention is an active fragment of the naturally occurring sequence of IL-11 or an active fragment of a sequence that is sufficiently homologous to the naturally occurring sequence of IL-11.

Active fragments of IL-11 generally have a sequence that is identical to or sufficiently homologous with IL-11 but includes fewer amino acids than the full length protein, and retains the ability to block thrombocytopenia, liver damage and/or liver damage-related inflammation. Specifically, an active fragment of IL-11, for purposes of the present invention, may be one that retains the ability to prevent, slow down, reduce or stop thrombocytopenia; prevent, slow down, reduce or stop liver damage; limit liver-damage-related inflammation; or any combination thereof, when administered to a subject. Typically, an active fragment may comprise a domain or motif of the full length protein having the activity. An active fragment can be of a polypeptide which is, for example, 10, 25, 50, 100, 150, 175, 177, 178, 180, 185, 190, 195, 200 or more amino acids in length. When applied to IL-11, the term “active fragment” refers to any peptide comprising an amino acid sequence sufficiently homologous to or derived from the amino acid sequence of naturally occurring IL-11, which includes fewer amino acids than the full length protein, and retains the ability to prevent, slow down, reduce or stop thrombocytopenia; prevent, slow down, reduce or stop liver damage; limit liver-damage-related inflammation; or any combination thereof, when administered to a subject.

Particular active fragments of IL-11 have amino acid sequences substantially identical to a portion of the amino acid sequence of the naturally occurring IL-11 sequence. Such substantially identical sequences contain significant number of amino acid residues that are (i) identical to, or (ii) conservative substitutions of aligned amino acid residues such that they include relevant structural domain and/or functional activity of IL-11. For example, amino acid sequences that contain a common structural domain showing at least about 60% or 65% identity, at least 75% identity, or at least 85%, 90%, 95%, 96%, 97%, 98%, 99%, or 100% identity with a relevant domain of IL-11 may be substantially identical.

Interleukin-11 to be used in the compositions and methods of the present invention may be obtained by any suitable method. Methods of producing polypeptides or proteins are well known in the art. For example, IL-11 can be obtained as a homogeneous protein purified from a mammalian cell line secreting it; or it can be chemically synthesized. Alternatively, interleukin-11 can be produced via recombinant techniques to enable large quantity production of pure, active IL-11 useful for therapeutic applications (as described in U.S. Pat. Nos. 5,215,895; 5,31,193; 5,700,664; 5,854,028 and 6,066,317, each of which is incorporated by reference in its entirety).

For example, IL-11 included in an inventive pharmaceutical composition or used in an inventive method of treatment may be produced in a host cell by recombinant DNA methods. Host cells may be mammalian or non-mammalian cells. Suitable mammalian cells include, but are not limited to, non-human mammalian tissue culture cells such as Chinese Hamster Ovary (CHO) cells, monkey COS cells, and mouse fibroblast NHI3T3 cells; and human tissue culture cells such as HeLa cells, HL-60 cells, kidney 293 cells, and epidermal S431 cells. Non-mammalian host cells include bacteria cells such as Escherichia coli, Bacillus subtilis, attenuated strains of Salmonella typhimurium, and the like; yeast cells such as Saccharomyces cerevisiae, Schizosaccharomyces pombe, Kluyveromyces strains, Candida, or any yeast strain capable of expressing proteins; insect cells such as Spodoptera frugiperda.

In certain embodiments, IL-11 included in an inventive pharmaceutical composition or used in an inventive method of treatment is human IL-11 produced in Escherichia coli (E. coli) by recombinant DNA methods. The protein produced by this method is 177 amino acids in length and differs from the 178 amino acid length of native IL-11 only in lacking the amino-terminal proline residue. This alteration was not found to result in measurable differences in bioactivity in either in vitro or in vivo (U.S. Pat. No. 6,066,317).

Recombinant human IL-11 produced by this method is called Oprelvekin (S. V. Sitaraman and A. T. Gewirtz, Curr. Opin. Invest. Drugs, 2001, 2: 1395-1400). Oprelvekin, is the active ingredient in NEUMEGA® (Wyeth), the first and only platelet growth factor commercially available so far. In November 1997, the FDA cleared Oprelvekin for the prevention of severe thrombocytopenia and the reduction of the need for platelet transfusions following myelosuppressive chemotherapy in susceptible patients with non-myeloid malignancies (J. A. Kaye, Stem Cells, 1996, 14 Suppl. 1: 256-260). NEUMEGA® (Oprelvekin) can help prevent progressively lower platelet counts caused by chemotherapy. In particular, treatment with NEUMEGA® may help cancer patients keep their chemotherapy planned dose on time (PDOT), thereby avoiding dose reduction and dose delays, and may help reduce the need for platelet transfusion. NEUMEGA® (Oprelvekin) has also shown potent thrombopoietic activity in animal models of compromised hematopoiesis, including moderately to severely myelosuppressed mice and non-human primates. In these models, NEUMEGA® improved platelet nadirs and accelerated platelet recoveries compared to controls.

IV. Methods of Treatment

In one aspect, the present invention relates to methods and/or systems for the management of drug-induced thrombocytopenia including thrombocytopenia resulting, at least partly, from drug-induced liver damage and thrombocytopenia resulting, at least partly, from drug-induced bone marrow destruction. Specifically, the invention provides methods and/or systems for alleviating thrombocytopenia (i.e., for preventing, reducing, slowing down or stopping thrombocytopenia).

In general, methods of prevention are aimed at delaying or preventing the onset of a medical condition. In such methods, therapy is typically administered prior to the onset of the condition for a prophylactic action. Methods of treatment, in general, are aimed at (1) slowing down or stopping the progression, aggravation, or deterioration of the symptoms of a medical condition; (2) bringing about amelioration of one or more symptoms of the condition; and/or (3) curing the condition. In such methods, therapy is typically administered after initiation of the condition, for a therapeutic action.

Methods of the present invention include the step of: administering a therapeutically effective amount of interleukin-11 to a subject in need thereof. Appropriate subjects or individuals receiving inventive therapy include humans or another mammals (e.g., mice, rats, rabbits, dogs, cats, cattle, swine, sheep, horses, or primates) that are or can be afflicted with, or are susceptible to, a disease or disorder (e.g., thrombocytopenia, liver damage or bone marrow destruction) but may or may not have the disease or disorder or symptoms of the disease or disorder. In some embodiments, the subject is sometimes a human patient.

Methods of the present invention often involve administration of a therapeutically effective amount of a particular agent. A therapeutically effective amount is an amount sufficient to achieve (in principle, for a subject of comparable characteristics, such as species, body type, size, extent of disease or disorder, degree or type of symptoms, history of responsiveness, and/or overall health) an intended biological or medical response or therapeutic benefit in a tissue, system or subject. For example, a desirable response may include one or more of: delaying or preventing the onset of a medical condition, disease or disorder, slowing down or stopping the progression, aggravation, or deterioration of the symptoms of the condition, bringing about ameliorations of the symptoms of the condition, and curing the condition.

A therapeutically effective amount of a chemotherapeutic is typically an amount sufficient to achieve an intended delay, reduction, or amelioration of tumor progress. A therapeutically effective amount of IL-11 may be different depending on the desired response. For instance, an amount of IL-11 effective to prevent thrombocytopenia may be different from an amount of IL-11 effective to treat thrombocytopenia, and either may be different from amounts to prevent or treat liver damage or bone marrow destruction. Similarly, an amount of IL-11 effective to prevent thrombocytopenia induced by a first drug may be different from an amount of IL-11 effective to prevent thrombocytopenia induced by a second, different drug, etc.

Also, it will be appreciated that, when combinations of therapeutic agents are administered, the amount of any individual agent required in the combination may be different from the amount required of that same agent to achieve its therapeutic effect alone. In some cases, synergies between or among therapeutic agents used in a combination may reduce amounts required; in other cases, inhibitory interactions may increase amounts required. Thus, in general, therapeutically effective amounts of a combination of agents may utilize different absolute amounts of the agents than constitute therapeutically effective amounts of the agents individually.

Methods of the present invention may be used to prevent the onset of thrombocytopenia (e.g., in a subject undergoing or intending to undergo therapy involving a therapeutic conjugate whose administration results in, or may result in thrombocytopenia). Alternatively, methods of the present invention may be used to treat thrombocytopenia (e.g., in a subject that has received and/or is receiving therapy involving a therapeutic conjugate whose administration results in thrombocytopenia). It will be appreciated that different platelet counts may warrant a thrombocytopenia diagnosis for different mammalian species. In humans, the platelet count in the circulating blood is normally between 150 and 400 million per milliliter of blood (or 150 to 400×10⁹/L).

Laboratory tests used to diagnose thrombocytopenia (or to assess the effects of the methods of treatment of the present invention) include full blood count. A cell count analysis may be performed manually, by viewing a slide prepared with a sample of the patient's blood under a microscope, or automatically, by using a automated analyzer (e.g., a Coulter Model S-plus instrument). A full blood count usually provides information on the concentrations of different cells present in the blood, including platelets.

A. Liver Damage

Certain embodiments of the present invention provide methods and compositions for administration to a subject suffering from or susceptible to thrombocytopenia resulting from drug-induced liver damage. A subject may be suffering from or susceptible to liver damage if that subject that has been diagnosed with liver damage (e.g., has been tested and found to have liver damage); is suspected of having liver damage (e.g., presents one or more symptoms indicative of liver damage, has one or more risk factors, or is being screened for liver damage); is clinically known to have a tendency to suffer from liver damage (e.g., has a history of liver damage); or has received, is receiving or is about to receive a treatment involving a therapeutic agent whose administration results in liver damage. Individuals that have previously undergone therapy for liver damage may also be considered to be suffering from or susceptible to liver damage.

Liver damage may include any injury, defect or dysfunction of the liver caused by a particular factor (e.g., a conjugate) or a combination of factors. For instance, liver damage includes, but is not limited to, degeneration or necrosis of liver parenchyma cells; mobilization, infiltration, activation of Kupffer cells, leukocytes, and the like; swelling of the liver; fibrosis of the liver tissue; as well as any biological reaction or condition that results from the damage (including, but not limited to, inflammation and splenomegaly).

In certain embodiments, the methods and pharmaceutical compositions of the present invention are used to prevent or treat drug-induced liver damage that results in thrombocytopenia. For example, the inventive methods and pharmaceutical compositions may be used to prevent the onset of drug-induced liver damage (e.g., in a subject to be submitted to a treatment involving a conjugate whose administration results in liver damage). Alternatively or additionally, inventive methods and pharmaceutical compositions may be used to treat drug-induced liver damage (e.g., in a subject that has received a treatment involving a conjugate whose administration results in liver damage).

Liver damage may be diagnosed, according to established techniques. For example, liver damage is often diagnosed using a set of clinical biochemistry laboratory blood assays designed to provide information about the state of the subject's liver. Since the liver produces most of the plasma proteins in the body, measuring the amount of total protein and/or the amount of albumin (the main constituent of total protein and a protein made specifically by the liver) in the blood gives information regarding the functioning state of the liver. When the liver is damaged, it may fail to produce blood clotting factors: the prothrombin time may be measured to diagnose disorders of blood clotting, usually bleeding, resulting from liver damage. Serum bilirubin concentration may also be measured as an indication of the patient's liver state. Bilirubin is the major breakdown product that results from the destruction of old red blood cells (as well as other sources). It is removed from the blood by the liver, chemically modified by a process called conjugation, secreted into the bile, passed into the intestine and to some extent reabsorbed from the intestine. Many different liver diseases and conditions can cause the serum bilirubin concentrations to be elevated. Blood assays may also be performed to measure one or more of alanine transaminase (ALT), alkaline phosphatase (ALP), aspartate transaminase (AST), and gamma glutamyl transpeptidase (GGT). ALT is an enzyme present in hepatocytes. When a hepatic cell is damaged, it leaks this enzyme into the blood, where it can be measured. ALT rises dramatically in acute liver damage, such as viral hepatitis or acetaminophen overdose. Elevations are often measured in multiples of the upper limit of normal (ULN). ALP is an enzyme present in the cells lining the biliary ducts of the liver. If there is an obstruction in the bile duct (e.g., gallstones), ALP levels in the plasma will rise. AST is similar to ALT in that it is another enzyme associated with liver parenchymal cells, that is raised in acute liver damage. GGT is an enzyme whose levels may be elevated with even minor, sub-clinical levels of liver dysfunction.

Alternatively or additionally, these laboratory tests for the diagnosis of liver damage may be used to assess the effects of the methods of the present invention.

In certain embodiments of the present invention, liver damage is associated with (i.e., includes, is accompanied by or results in) inflammation. Inflammation is a natural consequence of injury of adult tissue and the body's initial attempt at healing itself. During the early phase of inflammation, neutrophils and macrophages are attracted to the site of injury/damage. Once activated, they produce large amounts of reactive oxygen species (ROS) through an oxygen-consuming respiratory burst. One purpose of these cell products is to destroy damaged tissue, kill invading organisms and prevent infection. Despite this beneficial effect, prolonged production of high levels of ROS can impair healing and cause severe additional tissue damage and deterioration.

Methods and pharmaceutical compositions of the present invention may be used to prevent or limit inflammation associated with drug-induced liver damage. For example, inventive methods and pharmaceutical compositions may be used to limit the extent or degree of inflammation otherwise observed after administration of a conjugate producing liver damage.

The extent of inflammation and/or the effects of inventive methods and compositions on inflammation may be evaluated using any of a variety of suitable tests known in the art. Such tests include assays measuring the erythrocyte sedimentation rate (ESR), which serves as a convenient way to screen for any inflammatory process in the body; assays measuring C-reactive protein (CRP, whose production by the liver increases up to a thousand fold in response to insult); and assays measuring neutrophil counts.

B. Bone Marrow Destruction

Certain embodiments of the present invention relate to methods and compositions for administration to a subject suffering from or susceptible to thrombocytopenia resulting from drug-induced bone marrow destruction. A subject may be suffering from or susceptible to bone marrow destruction if that subject has been diagnosed with bone marrow destruction (e.g., has been tested and found to present bone marrow damage, defect or dysfunction); is suspected of having bone marrow destruction (e.g., presents one or more symptoms of bone marrow destruction); or has received, is receiving or is about to receive a treatment involving a conjugate whose administration results in bone marrow destruction.

Bone marrow destruction may include any damage, defect or dysfunction of the bone marrow caused by a particular factor (e.g., conjugate) or a combination of factors that results in decreased or defective platelet production. For instance, bone marrow destruction includes, but is not limited to, degeneration or necrosis of megakaryocytes, depressed production of megakaryocytes, and alteration or damage of bone marrow producing an unfavorable environment for platelet production from megakaryocytes.

In certain embodiments, the inventive methods and pharmaceutical compositions are used to prevent or treat drug-induced bone marrow destruction that results in thrombocytopenia. For example, methods and pharmaceutical compositions of the present invention may be used to prevent drug-induced bone marrow destruction (e.g., in a subject to be submitted to a treatment involving a conjugate whose administration results in bone marrow destruction) or to treat drug-induced bone marrow destruction (e.g., in a subject that has received a treatment involving a conjugate whose administration results in bone marrow destruction).

Bone marrow destruction may be diagnosed according to established techniques. For example, bone marrow destruction may be diagnosed by assessing the cellularity and morphology of residual erythroid cells in a bone marrow aspirate or biopsy. Bone marrow activity can also be determined by radiographic methods or imaging methods including magnetic resonance imaging (MRI) and positron emission tomography (PET).

C. Selection of Subjects

In certain embodiments, subjects suitable to receive a treatment according to the present invention include individuals suffering from drug-induced thrombocytopenia; individuals clinically known to have a tendency to suffer from drug-induced thrombocytopenia; individuals that have received, are receiving or are about to receive a treatment involving a conjugate therapeutic agent whose administration results in thrombocytopenia. Suitable subjects may or may not have previously received traditional treatment for the condition.

Before administration of an inventive therapy or composition, a subject may be tested for thrombocytopenia, liver damage, inflammation, and/or bone marrow destruction, using one or more of the methods described above. The same or similar methods may be used to determine the effects of the inventive treatment/pharmaceutical composition on the subject.

Subjects suffering from or susceptible to drug-induced thrombocytopenia may be suffering from cancer or a cancerous condition. In some embodiments, thrombocytopenia in the subject results, at least partly, from chemotherapeutic conjugate-induced liver damage. Alternatively or additionally, thrombocytopenia results, at least partly, from chemotherapeutic conjugate-induced bone marrow destruction.

In general, cancer or cancerous condition refers to or describes a physiological condition in mammals that is typically characterized by unregulated cell growth. Examples of cancers include, but are not limited to, carcinoma, lymphoma, blastoma, sarcoma, and leukemia. More particularly, examples of such cancers include squamous cell carcinoma, small-cell lung cancer, non-small cell lung cancer, pancreatic cancer, glioblastoma multiform, melanoma, multiple myeloma, non-Hodgkin's lymphoma, esophageal/oral cancer, cervical cancer, ovarian cancer, endometrial cancer, prostate cancer, bladder cancer, hepatoma, breast cancer, colon and rectal cancer, bone cancer, renal cancer, myeloid, lymphocytic, myelocytic, and lymphoblastic leukemias, and head and neck cancer, to name a few.

In some embodiments, subjects suitable to receive an inventive therapy and/or a pharmaceutical composition include cancer patients suffering from or susceptible to thrombocytopenia. Such cancer patients may include individuals diagnosed with cancer (e.g., tested and found to have cancer), individuals suspected of having cancer (e.g., presenting one or more symptoms indicative of cancer, having one or more risk factors, or being screened for cancer). Alternatively or additionally, cancer patients may include individuals that have previously undergone therapy for cancer.

D. Dosage and Administration

Administration of IL-11 (and/or other agent), according to methods of the present invention, may consist of a single dose or a plurality of doses over a period of time. Administration of interleukin-11 prior to administration of CMC-544 results in prevention of CMC-544-related liver damage and thrombocytopenia (see Example 1), and in reduction of CMC-544-related inflammation (see Example 2). Accordingly, in certain embodiments, IL-11 is administered prior to administration of the conjugate that produces thrombocytopenia. Alternatively or additionally, IL-11 may be administered concurrently with administration of the conjugate and/or following administration of the conjugate.

Inventive administrations may be carried out in any convenient manner such as by injection (subcutaneous, intravenous, intramuscular, intraperitoneal, or the like) or oral administration.

Depending on the route of administration, effective doses may be calculated according to the body weight, body surface area, or organ size. Optimization of the appropriate dosages can readily be made by one skilled in the art, for example, in light of pharmacokinetic data observed in pre-clinical studies or human clinical trials. The final dosage regimen will generally be determined by the attending physician, who may consider various factors which modify the action of the drugs, e.g., the drug's specific activity, the severity of the damage and the responsiveness of the patient, the age, condition, body weight, sex and diet of the patient, the severity of any present infection, time of administration, and other clinical factors. As studies are conducted, further information will emerge regarding the appropriate dosage levels and duration of treatment.

It will also be appreciated that, in the methods of the present invention, IL-11 and/or other therapeutic agents (e.g., conjugates) can be employed in combination therapies (i.e., can be administered concurrently with, prior to, or subsequent to one or more desired therapies of medical procedures). The particular combination of therapies (therapeutics or procedures) to employ in a combination regimen will usually take into account compatibility of the desired therapeutics and/or procedures and the desired therapeutic effect to be achieved.

For example, in those embodiments where the conjugate is used in the treatment of cancer, methods of the present invention can be employed in combination with other procedures including surgery, radiotherapy (e.g., γ-radiation, neuron beam radiotherapy, electron beam radiotherapy, proton therapy, brachytherapy, and systemic radioactive isotopes), endocrine therapy, hyperthermia and cryotherapy.

Alternatively or additionally, methods of the present invention can be employed in combination with other agents, for example to attenuate one or more adverse effects (e.g., antiemetics, pain relievers, anti-nausea drugs), other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies, see The Merck Manual, 17^{th} Ed. 1999, the cancer therapeutics sections of which are hereby incorporated by reference.

Methods of the present invention can also be employed together with one or more combinations of cytotoxic agents as part of a treatment regimen, wherein the combination of cytotoxic agents is selected, for example, from CHOPP (cyclophosphamide, doxorubicin, vincristine, prednisone, and procarbazine); CHOP (cyclophosphamide, doxorubicin, vincristine, and prednisone); COP (cyclophosphamide, vincristine, and prednisone); CAP-BOP (cyclophosphamide, doxorubicin, procarbazine, bleomycin, vincristine, and prednisone); m-BACOD (methotrexate, bleomycin, doxorubicin, cyclophosphamide, vincristine, dexamethasone, and leucovorin); ProMACE-MOPP (prednisone, methotrexate, doxorubicin, cyclophosphamide, etoposide, leucovorin, mechloethamine, vincristine, prednisone, and procarbazine); ProMACE-CytaBOM (prednisone, methotrexate, doxorubicin, cyclophosphamide, etoposide, leucovorin, cytarabine, bleomycin, and vincristine); MACOP-B (methotrexate, doxorubicin, cyclophosphamide, vincristine, prednisone, bleomycin, and leucovorin); MOPP (mechloethamine, vincristine, prednisone, and procarbazine); ABVD (adriamycin/doxorubicin, bleomycin, vinblastine, and dacarbazine); MOPP (mechloethamine, vincristine, prednisone and procarbazine) alternating with ABV (adriamycin/doxorubicin, bleomycin, and vinblastine); MOPP (mechloethamine, vincristine, prednisone, and procarbazine) alternating with ABVD (adriamycin/doxorubicin, bleomycin, vinblastine, and dacarbazine); ChlVPP (chlorambucil, vinblastine, procarbazine, and prednisone); IMVP-16 (ifosfamide, methotrexate, and etoposide); MIME (methyl-gag, ifosfamide, methotrexate, and etoposide); DHAP (dexamethasone, high-dose cytaribine, and cisplatin); ESHAP (etoposide, methylpredisolone, high-dose cytarabine, and cisplatin); CEPP(B) (cyclophosphamide, etoposide, procarbazine, prednisone, and bleomycin); CAMP (lomustine, mitoxantrone, cytarabine, and prednisone); CVP-1 (cyclophosphamide, vincristine, and prednisone), ESHOP (etoposide, methylpredisolone, high-dose cytarabine, vincristine and cisplatin); EPOCH (etoposide, vincristine, and doxorubicin for 96 hours with bolus doses of cyclophosphamide and oral prednisone), ICE (ifosfamide, cyclophosphamide, and etoposide), CEPP(B) (cyclophosphamide, etoposide, procarbazine, prednisone, and bleomycin), CHOP-B (cyclophosphamide, doxorubicin, vincristine, prednisone, and bleomycin), CEPP-B (cyclophosphamide, etoposide, procarbazine, and bleomycin), and P/DOCE (epirubicin or doxorubicin, vincristine, cyclophosphamide, and prednisone).

Alternatively or additionally, methods of the present invention can be employed together with therapies involving administration of one or more of bioactive agents selected from the group consisting of antibodies, growth factors (e.g., Tumor-Necrosis Factor (TNF), Colony Stimulating Factor (CSF), Granulocyte-Colony Stimulating Factor (G-CSF) or Granulocyte-Macrophage Colony Stimulating Factor (GM-CSF)), hormones (e.g., estrogens, androgens, progestins, and corticosteroids), cytokines, anti-hormones, xanthines, interleukins (e.g., IL-2), and interferons.

E. Mechanisms

Kinetics of platelet decrease provide a rough guide to the mechanism of thrombocytopenia; although the kinetics of platelet reduction is a highly variable criterion and can be dependent on the chemical entity used. For example, mice treated with cyclophosphamide have been shown to develop a platelet nadir on day 3 (G. Hangoc et al., Blood, 1993, 81: 965-972); and mice treated with carboplatin and irradiation exhibit a nadir on day 9 (J. P. Leonard et al., Blood, 1994, 83: 1499-1506). Based on the present Examples, carboplatin alone gave a nadir of 11. All of these agents affect platelet production, but they have very different effects on the timing of the platelet nadir. Similar effects have been reported in monkeys. Carboplatin was found to give a platelet nadir on day 13-14 (F. J. Schlerman et al., Stem Cell, 1996, 14: 517-532), but ACNU (i.e., 3-[(4-amino-2-methyl-5-pyrimidinyl)methyl]-1-(2-chloroethyl)-1-nitrosourea hydrochloride) does not produce a nadir until day 21 (M. Saitoh et al., J. Interferon and Cytokine Res., 2000, 20: 539-545). In humans, CHOP has been shown to produce a nadir around day 9. Despite the differences, thrombocytopenia produced by these classical chemotherapeutic agents, all of which are bone marrow toxins, is thought to result from disruption of platelet production in the bone marrow.

The mechanisms of thrombopoiesis are also very complex and can be impacted in many different ways. Many chemotherapeutic agents have direct effects on megakaryocytes and on human megakaryocytic progenitors (CFU-megs). Since megakaryocytes do not divide, they may not necessarily stop producing platelets immediately when hit by a cycle-specific toxin. If the effects of the chemical agent are directed more toward progenitors and early-megakaryocytes, the effect on peripheral platelets is slower and the nadir occurs later. If the effects are directly on megakaryocytes and affect proteins as well as DNA, the effects on circulating platelets can occur more rapidly.

Alternatively, CMC-544 (and other similar protein conjugates) may have a clear impact on the liver (transaminases released) and on TPO production in the liver (reduced TPO levels). TPO production seems to slow very soon after CMC-544 administration. This would be expected to result in an immediate reduction in stimulation of megakaryocytes and an associated reduction of platelet production. However, since the half life of platelet is around 5 days in humans, the kinetics suggest that this is not the sole mechanisms of thrombocytopenia.

The effects of oprelvekin (IL-11) observed in the present Examples are consistent with its known effects as a liver protective agent (in animals, see for example, W. L. Trepicchio et al., Toxicol. Pathol., 2001, 29: 242-249; and in humans, see for example, R. Ghalib et al., Hepatology, 2003, 37: 1165-1171). Its ability to ameliorate the platelet drop associated with CMC-544 treatment, when IL-11 is given prophylactically but not when it is given concomitantly, is also consistent with the known kinetics of platelet products induced by IL-11. Administration of IL-11 6 hours after CMC-544 administration may have been too late to have full protective effect on the liver, but it may have some positive effect nevertheless.

Thrombocytopenia associated with treatment using CMC-544 (and other similar protein conjugates) may result, at least in part, from disruption of TPO production secondary to liver damage and is distinct from that caused by conventional chemotherapeutic agents that disrupt platelet production by damaging bone marrow cells.

V. Pharmaceutical Compositions and Kits

In another aspect, the present invention provides pharmaceutical compositions comprising a therapeutically effective amount of interleukin-11 (IL-11) and at least one physiologically acceptable carrier or excipient.

A physiologically acceptable carrier or excipient generally is a carrier medium or an excipient that does not block the effectiveness of the biological activity of the active ingredient(s) of the composition and that is not excessively toxic to the host at the concentrations at which it is administered. The term includes solvents, dispersion media, coatings, antibacterial agents, isotonic agents, absorption delaying agents, and the like. The use of such media and agents for the formulation of pharmaceutically active substances is well known in the art (see, for example, “Remington's Pharmaceutical Sciences”, E. W. Martin, 18^{th} Ed., 1990, Mack Publishing Co.: Easton, Pa., which is incorporated herein by reference in its entirety).

In certain embodiments, IL-11 is the only active ingredient in an inventive pharmaceutical composition. In other embodiments, the pharmaceutical composition further comprises one or more other therapeutic agents (e.g., one or more conjugates). In still other embodiments, the pharmaceutical composition further comprises a combination of therapeutic agents. One or more conjugates included in an inventive pharmaceutical composition may induce liver damage and/or bone marrow destruction.

A. Formulations

In certain embodiments, a pharmaceutical composition of the present invention is administered in such amounts and for such time as necessary to achieve a desired result. For example, a pharmaceutical composition can be administered in such amounts and for such time that it prevents, reduces, slows down or stops drug-induced thrombocytopenia in a subject (e.g., thrombocytopenia resulting from administration of a conjugate). More specifically, an inventive pharmaceutical composition can be administered in such amounts and for such time that it prevents, reduces, slows down or stops abnormally high destruction of circulating platelets and/or decreased platelet production by the bone marrow.

A pharmaceutical composition of the present invention may also be administered in such amounts and for such time that it prevents, reduces, slows down or stops thrombocytopenia resulting, at least partly, from drug-induced liver damage. Alternatively or additionally, a pharmaceutical composition may be administered in such amounts and for such time that is prevents, reduces, slows down or stops thrombocytopenia resulting, at least partly, from drug-induced bone marrow destruction.

In certain pharmaceutical compositions, interleukin-11 (IL-11), one or more conjugate(s) and one or more physiologically acceptable carriers or excipients are combined in one or more preparations for simultaneous or sequential administration of IL-11 and the conjugate(s). More specifically, an inventive composition may be formulated in such a way that IL-11 and the conjugate(s) can be administered at the same time or independently from each other (e.g., the composition can comprise one or more preparations in individual containers).

Pharmaceutical compositions, according to the present invention, may be administered using any amount and any route of administration effective for preventing, slowing down, reducing or stopping thrombocytopenia that would otherwise be observed in the absence of IL-11 administration.

As already mentioned above, the exact amount of pharmaceutical composition to be administered may vary from subject to subject, depending on the species, age, and general condition of the subject, the severity of the condition, the particular therapeutic agent, its mode of administration, the severity of thrombocytopenia and/or liver damage or bone marrow destruction it induces, and the like.

Desirable optimal pharmaceutical formulations can be determined depending upon the route of administration and desired dosage. Such formulations may influence the physical state, stability, rate of in vivo release, and rate of in vivo clearance of the administered compounds.

Pharmaceutical compositions of the present invention may be formulated in dosage unit form for ease of administration and uniformity of dosage. The expression “dosage unit form”, typically refers to a physically discrete unit of IL-11 alone, conjugate alone, or combination of IL-11 and conjugate appropriate for the patient to be treated. It will be understood, however, that the total daily usage of the compositions of the present invention will generally be decided by an attending physician within the scope of sound medical judgment.

After formulation with one or more appropriate physiologically acceptable carriers or excipients in a desired dosage(s), pharmaceutical compositions of the present invention can be administered to humans or other mammals by any suitable route. For example, pharmaceutical compositions of the present invention may be administered orally, parenterally, intravenously, intraperitoneally, intramuscularly or subcutaneously, depending on the condition being treated (e.g., thrombocytopenia resulting from bone marrow destruction or thrombocytopenia resulting from liver damage) by administration of the at least one therapeutic agent.

Injectable preparations, for example, sterile injectable aqueous or oleaginous suspensions, may be formulated according to established procedures, for example, using suitable dispersing or wetting agents, and suspending agents. Sterile injectable preparations may also be sterile injectable solutions, suspensions or emulsions in a non-toxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or di-glycerides. Fatty acids such as oleic acid may also be used in the preparation of injectables.

Inventive injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished, for example, through the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions which are compatible with body tissues.

Liquid dosage forms for oral administration include, but are not limited to, pharmaceutically acceptable emulsions, microemulsions, solutions, suspensions, syrups, and elixirs. In addition to the active ingredients, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cotton seed, ground nut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound may be mixed with at least one inert, pharmaceutically acceptable excipient or carrier such as sodium citrate or dicalcium phosphate and/or (a) fillers or extenders such as starches, lactose, sucrose, glucose, mannitol, and silicic acid; (b) binders such as, for example, carboxymethylcellulose, alginates, gelatin, polyvinylpyrrolidinone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin, (f) absorption accelerators such as quaternary ammonium compounds; (g) wetting agents such as, for example, cetyl alcohol and glycerol monostearate; (h) absorbents such as kaolin and bentonite clay; and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

Active compounds (e.g., IL-11 and/or one or more conjugates) can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is common practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

B. Kits

In still another aspect, the present invention provides pharmaceutical packs or kits comprising one or more containers (e.g., vials, ampoules, test tubes, flasks or bottles) containing one or more ingredients of the inventive pharmaceutical compositions, for example, allowing for the simultaneous or sequential administration of interleukin-11 and conjugate(s).

In certain embodiments, an inventive kit includes one or more additional approved therapeutic agents for use as a combination therapy (e.g., one or more anti-cancer drugs as described above). Optionally associated with such container(s) can be a notice in the form prescribed by a governmental agency regulating the manufacture, use or sale of pharmaceutical products, which notice reflects approval by the agency of manufacture, use or sale for human administration.

Different ingredients may be supplied in solid (e.g., lyophilized) or liquid form. Each ingredient will generally be suitable as aliquoted in its respective container or provided in a concentrated form. Kits may also include media for the reconstitution of lyophilized ingredients. The individual containers of the kit are preferably maintained in close confinement for commercial sale.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Effect of IL-11 on CMC-544 induced Thrombocytopenia in Nude Mice

Experimental Design:

The effect of IL-11 (NEUMEGA®) on CMC-544-induced thrombocytopenia is shown in the present example. The initial IL-11 dose was administered i.p. daily (250 μg/kg) beginning up to 2 days before, or BID (125 μg/kg) beginning up to 8 hours after CMC-544 administration (considered Day 0). IL-11 was given daily for up to 8 days post CMC-544 administration. On Day 0, mice were bled for baseline platelet values and then dosed with vehicle or CMC-544 at 4 μg/mouse i.p. Higher or lower doses of CMC-544 were also administered. Blood was sampled at various time points up to 3 days post drug administration.

Procedures:

A 25 gauge needle was inserted into the tail vein of the mouse and then withdrawn allowing for a drop of blood to seep out. A 5 μL sample of blood was collected for analysis. Platelet values were quantitated using a dual threshold Beckman Coulter Z1 Particle Counter (Fullerton, Calif.). Threshold values were set to measure mouse platelets according to manufacturer specification.

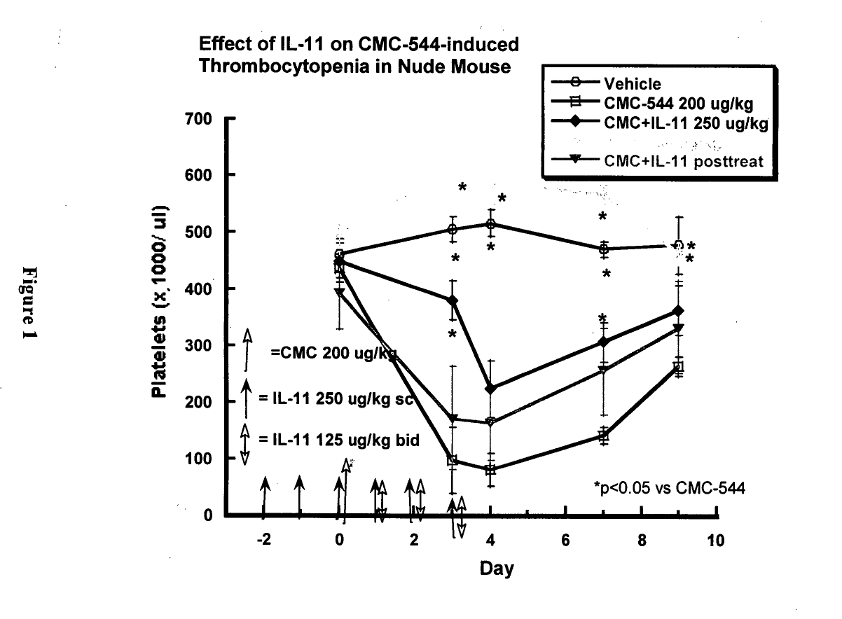

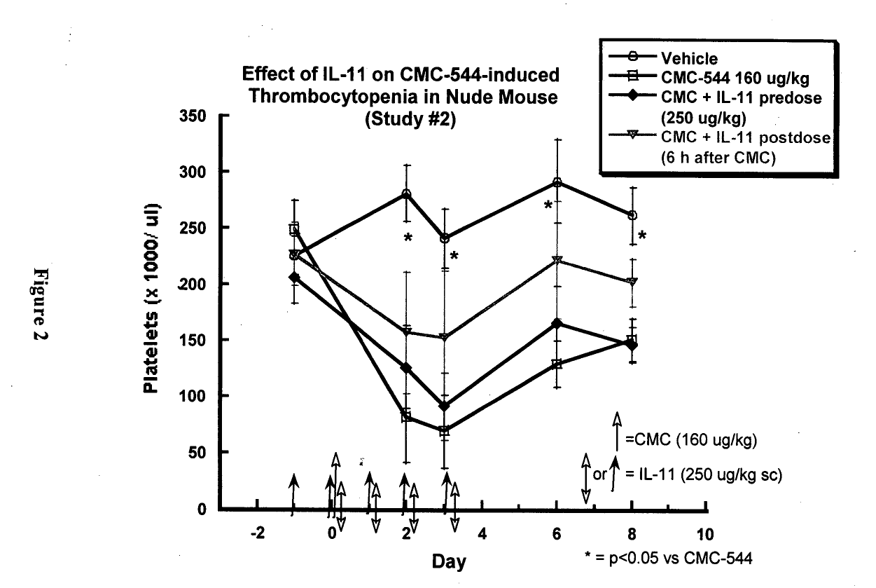

As shown on FIGS. 1 and 2, administration of IL-1 prior to administration of CMC-544 prevents CMC-544-induced reduction in platelet count in nude mice.

### Example 2

### Effects of IL-11 on CMC-544 induced Thrombocytopenia in Monkeys

A—First Study

Experimental Design:

Ten (10) monkeys (cynomolgus macaques) were bled initially on Day −9 in order to select eight (8) of them to be put on study that have the more normal blood values. The selected monkeys were divided into 2 groups of 4 each. One group of test monkeys was pre-dosed with IL-11 for 5 days prior to receiving CMC-544. The other group of monkeys (or control monkeys) was administered a vehicle control of sterile saline. This provided the appropriate control for the stress involved in dosing the monkeys with IL-1 in order to discern between potential vehicle and/or IL-11 side effects (i.e., effects on blood or hematology parameters) if they were to occur. Both groups received CMC-544 on Day 1. Dosing with IL-11 in the test group also took place on the day of and continue for 4 days after CMC-544 administration. The test group received a total of 10 doses of IL-1. Both groups of monkeys were monitored for 6 days after CMC-544 at which time they were euthanized.

Blood was drawn for analysis on Day −11 (pre-test), Day −5 (time of first IL-11 administration), Day 1 (time of CMC-544 administration), Days 3, 4, 5 (time of platelet nadir for CMC-544), and Day 7 (end of the study). For each day that requires a blood sample collection and drug administration, blood was drawn before IL-11 or CMC-544 administration. On Day 1, IL-11 was administered immediately after CMC-544 administration.

More specifically, the test group of monkeys were submitted to the following procedures: on Day −11: drawing of 6 mL of blood; on Day −5: drawing of 2 mL of blood, administration of IL-11; on Day −4: administration of IL-11; on Day −3: administration of IL-1; on Day −2: administration of IL-11; on Day −1: administration of IL-11; on Day 1 (drawing of 2 mL of blood, administration of IL-11 and CMC-544); on Day 2: administration of IL-11; on Day 3: drawing of 2 mL of blood, administration of IL-11; on Day 4: drawing of 6 mL of blood, administration of IL-11; on Day 5: drawing of 2 mL of blood, administration of IL-11; on Day 7: drawing of 6 mL of blood, euthanasia, and necrospy.

Procedures:

Drug Administration: IL-11 was administered at 125 μg/kg (as a solution) by subcutaneous injection. IL-11 was reconstituted fresh with sterile water and administered within 3 hours of reconstitution. The dose volume was 0.25 mL/kg. IL-11 was administered after any scheduled blood samples were taken. CMC-544 was administered at 25 μg/kg (dose based on Calicheamicin DMH content) intravenously as a solution. Dose volume was 1 mL/kg. Monkeys were anesthetized with ketamine (10 mg/kg) before iv injection of CMC-544 through an indwelling catheter. CMC-544 was administered after any scheduled blood samples were taken and immediately before IL-11 administration.

Blood Sampling: On Days −11, 4 and 7, a total of 6 mL of blood were collected according to the following procedure: 3 mL were collected in a red top tube (serum) for clinical chemistry analysis and 3 mL in a lavender top tube (EDTA anti-coagulant) for CBC (Complete Blood Count). On Days −5, 1, 3, and 5, 2 mL of EDTA treated blood (in lavender top tubes) were collected for platelet determinations.

Total blood collected per monkey were 8 mL the first week, 12 mL the second week, and 6 mL the third week. The total blood volume of a monkey is estimated to be 5.4% of body weight (54 mL/kg). Therefore, monkeys weighting>2.5 kg had less than 10% of blood volume withdrawn per week.

Ill Effects:

Monkeys were monitored daily for health and a staff veterinarian was notified of their condition if ill effects were observed. Food consumption was monitored daily.

IL-11 (125 mg/kg s.c.) has been previously administered to monkeys with no adverse events reported (F. J. Schlerman et al., “The effect of rhIL-11 on Platelet Aggregation in vitro and ex vivo following Subcutaneous Administration to Non-Human Primates for 14 Days”, Genetics Institute Report #P98034-14; A. G. Bree et al., “Thrombopoietic Activity of Recombinant Human Interleukin-11 Alone or in Combination with Recombinant Human Granulocyte-Colony Stimulating Factor in a Novel Myelosuppressed Non-Human Primate Model”, Genetics Institute Report #P99029-14; A. G. Bree et al., “Thrombopoietic Activity of a Normal or Short Course of Recombinant Human Interleukin-11 in Combination with a Normal or Delayed Course of Recombinant Human Granulocyte-Colony Stimulating Factor in a One-Cycle Myelosuppressed Non-Human Primate Model, Genetics Institute Report #P99030-14).

Previous studies with CMC-544 at the dose of 25 mg/kg reported reduced food consumption, emesis (75% of monkeys at day 4 post CMC-544 administration) and fecal alterations such as liquid, mucoid, soft and/or reduced feces (beginning day 4 through day 7 in ≧50% of monkeys). Fresh fruit and anti-diarrheal agents (Kao-pectate) may be provided for inappetance and for fecal changes, respectively. Monkeys exhibiting unanticipated adverse effects (other than the 3 symptoms reported above) were reported to the attending veterinarian for consultation.

Euthanasia:

Monkeys were killed by an overdose of barbiturates (100 mg/kg i.v.).

Necropsy:

Post-mortem tissue samples of liver, bone marrow (stern brae), lung (a section from each side of the lung), and spleen were collected and fixed in 10% formalin. Bone marrow smears were collected from an open rib.

Hematology:

Hematology and coagulation parameters were evaluated twice at pretest and dosing phase days 1, 3, 4, 5, and 7. Mean platelet volume (MPV) was evaluated during these dosing phase intervals only. In animals administered CMC-544 only, compound-related alterations in individual animals relative to their pretest values occurred in counts of platelets (PLT), neutrophils (NEU), monocytes (MONO), and eosinophils (EOS). In animals administered rhuIL-11 (NEUMEGA®), rhuIL-11-related changes were observed in parameters of red cell mass (hemoglobin, HGB, hematocit, HCT, and red blood cell count, RBC).

These decreases in PLT (31% to 69% relative to the first pretest count) were observed in all CMC-544 only-administered animals on dosing phase days 3, 4, 5 and (except for one animal) day 7, with the nadir generally observed at day 4 or 5 post-CMC-544 dosing. In these same animals, slight increases in PLT (30% to 126%) on dosing phase day 1 preceded these subsequent decreases. In contrast, all monkeys given rhuIL-11 prior to CMC-544, had slightly to mildly increased PLT at all dosing phase intervals (48% to 272%, relative to the first pretest value). These increases were peaked at day 4, 5 or 7 in these animals. These differences in PLT between groups support that rhuIL-11 pre-treatment prevents or ameliorates CMC-544-related platelet count decreases.

In males administered rhuIL-11 pretreatment, MPV was also generally greater at all dosing phase intervals than that of males administered CMC-544 alone. However, this pattern was not seen with the single female administered rhuIL-11 pretreatment compared with that given CMC-544 alone; thus, no conclusion pertaining to compound-related effects on MPV were made.

In all animals administered CMC-544 only, mild to moderate increases in NEU above pretest counts (85% to 212% relative to the first pretest value) were observed at one or more dosing phase intervals. Similarly, increases above pretest counts in MONO (39% to 386%) and EOS (50% to 538%, relative to the first pretest value) occurred during the dosing phase in these same animals. In contrast, dosing phase increases above pretest values occurred in only 1 of 4 animals given rhuIL-11 pretreatment, (the single female) in NEU (164% to 284%) and EOS (143%) at day 4 and/or 5, and in 2 of 4 animals (male or female) given rhuIL-11 pretreatment in MONO (93% to 329%). These differences between groups in incidence of increased leukocyte counts suggests amelioration or partial prevention of an inflammatory response to animals given rhuIL-11 prior to CMC-544. The change in NEU correlated with slight increased extramedullary hematopoiesis in the spleen in some individual animals given CMC-544 alone.

In all animals given rhuIL-11 prior to CMC-544, decreases in parameters of red cell mass (hemoglobin, hematocrit, red blood cell count) were observed at most to all dosing phase intervals (males: 12% to 32%; female: 22% to 42% relative to the first pretest values). These changes generally exceeded those at most intervals for animals given CMC-544 alone and were an anticipated effect of high dosage rhuIL-1 in monkeys. No notable differences in reticulocyte counts or red cell indices between animals given CMC-544 with or without rhuIL-11, and no correlation between these decreases in red cell mass and histologic bone marrow hypocellularity were observed.

Differences in other hematology parameters in rhuIL-11 administered animals compared with CMC-544 only-administered animals were not noteworthy due to their broadly overlapping absolute values and/or magnitude.

Clinical Chemistry:

Serum chemistry parameters were evaluated once at pretest and days 4 and 7. In animals administered CMC-544 only, compound-related increases (relative to pretest values) occurred in alanine aminotransferase (ALT). In one animal administered CMC-544 alone, and all animals administered rhuIL-11 (NEUMEGA®), increases in alkaline phosphatase (AP) also occurred; these compound-related increases were considered partly related to rhuIL-11 pretreatment. In all animals, decreases in albumin (ALB) occurred; the magnitude of these decreases was consistently greater with, and related to rhuIL-11 administration.

The increases in ALT (68% to 750%) occurred in 3 of 4 animals given CMC-544 only. These increases occurred at days 4 and/or 7 relative to each animal's pretest concentration and suggested minor to mild CMC-544-related liver injury. Supporting the occurrence of liver injury and correlating with the changes in ALT in these animals were microscopic mild multifocal hepatocellular necrosis (primarily centrilobular) and single cell necrosis in the monkey with the greatest increase in ALT (8.5 fold, female), and mild single cell necrosis in another animal (male) with a slight (1.7-fold) increase in ALT. The occurrence of these ALT increases in animals administered CMC-544 alone and not in those given rhuIL-11 pretreatment provided limited (due to small number of animals to evaluate) support for amelioration of liver injury with rhuIL-11 pretreatment.

Increases in AP occurred at both dosing phase intervals (relative to pretest concentrations) in one animal administered CMC-544 (122% to 242%) and all animals given rhuIL-11 pretreatment (276% to 589%). The greater incidence and magnitude of AP increase in rhuIL-1 administered animals and CMC-544 administered animals indicated the change is partly rhuIL-1-related. Similar increases in AP in monkeys following rhuIL-11 administration has been previously observed.

Decreases in ALB that were mild to moderate (17% to 45%) occurred at both dosing phase intervals in all animals administered rhuIL-11 pretreatment. These decreases corresponded with the decreases in red cell mass in these animals, as previously reported in monkeys given rhuIL-11. In contrast, decreases in ALB occurred in only some animals administered CMC-544 alone, and at one or both dosing phase intervals and were slight to mild (up to 24%). The greater incidence and magnitude of these decreases in animals administered rhuIL-11 relative to those given CMC-544 alone indicated the decrease is partly related to rhuIL-11 and notably exacerbated or superseded with rhuIL-11 administration in animals given CMC-544.

Differences in other serum chemistry values in compound-administered animals compared with controls were attributed to random variation due to their minor magnitude, direction, absence of a dosage-related pattern, and/or general overlap of individual values.

B—Second Study

A second study was carried out, the experimental design of which is presented in Table 1. Briefly, 8 female monkeys were used, 4 of them were administered a single dose i.v. of CMC-544 (on day 1; 25 μg/kg) and the other 4 received a single dose i.v. of CMC-544 (on day 1; 25 μg/kg) and 5 subcutaneous doses of NEUMEGA (125 μg/kg/day) on Days 1-5. Hematology studies were performed, as described above, twice prestudy and on dosing days 1 through 8, day 10, day 12 and day 14 (always prior to NEUMEGA administration). Coagulation and serum chemistry tests were performed, as described above, twice prestudy and on dosing days 7 and 14.

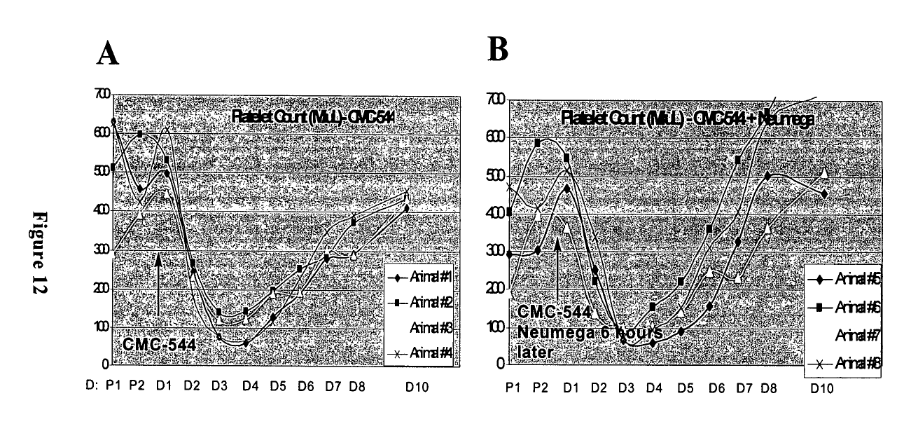

FIG. 12 shows the changes in platelet counts observed in both groups of monkeys. The onset and magnitude of platelet count decreases were found to be comparable between animals administered CMC-544 with or without NEUMEGA. Nadir occurred at post-dose day 3 or 4 (CMC-544 alone: 70% to 87%; CMC-544+NEUMEGA: 75% to &4% below last pretest value). Essentially complete resolution of these decreases (within 90% of the second pretest value) occurred by day 7 or 8 in all animals administered CMC-544+NEUMEGA, but only one animal given CMC-544 alone.

TABLE 1
Experimental Design

| Dosage | | Calicheamicin^{a} | NEUMEGA | Protein^{b} | Animal |
|---|---|---|---|---|---|
| Group | | (μg/kg/day) | (μg/kg/day) | (mg/m²) | Numbers |
| 1 | CMC-544^{a} | 25.0 | 0 | 4.28 | 1-4 |
| 2 | CMC-544^{a} + | 25.0 | 125 | 4.28 | 5-8 |
| | NEUMEGA^{c,d} | | | | |

^{a}Calicheamicin equivalents (˜82.5 μg/mg protein).
^{b}FDA mg/m2 conversion: http://www.fda.gov/cder/cancer/animalframe.htm
^{c}Dose Volume 1 mL/kg (CMC-544 administered on Day 1, ˜9 a.m).
^{d}0.025 mL/kg NEUMEGA administered once daily (˜3 p.m), days 1-5.

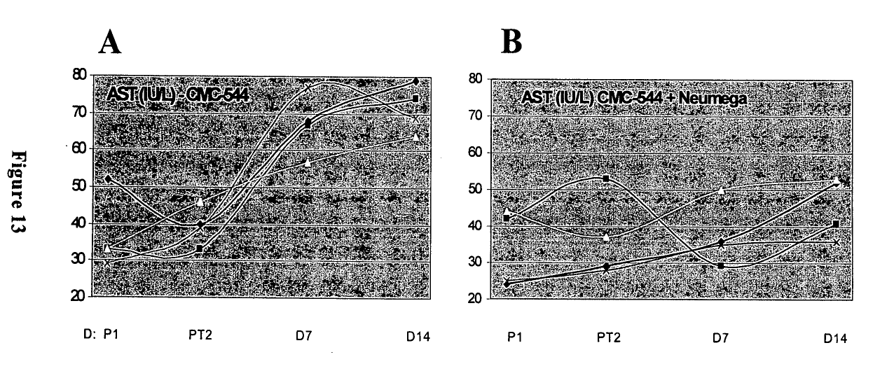

A slight increase in AST was observed at day 14 in all animals, and at day 7 in most animals administered CMC-544 alone (39-124% above last pre-study value) (See FIG. 13). The corresponding increase in ALT occurred in 2 of these four animals (82%-198%). Increases in these liver-associated serum enzymes that were of generally lower magnitude occurred at these intervals in 1 or 2 of 4 animals administered CMC-544+NEUMEGA (AST: 35-79%; ALT: 66%).

A slight to mild decrease in reticulocyte count was observed in all animals (8-56%) generally on days 3-5. These decreases were associated with slight to mild decreases in red cell mass (up to 22%) beginning on day 4. The incidence and onset between animals administered CMC-544 alone and animals administered CMC-544+NEUMEGA were similar in magnitude, and thus, are considered to be primarily CMC-544-related (as well as partly blood collection-related).

A slight to mild increase in GLOB was observed for both groups of animals (CMC-544 alone: 17-42%; CMC-544+NEUMEGA: 19-52%) at both day 7 and day 14 without a consistent pattern of resolution in either group.

A transient moderate increase in fibrinogen was observed at day 7 in all animals administered CMC-544+NEUMEGA (197% to 327% above pre-study values. These fibrinogen increases showed rapid partial resolution relative to pretest values by day 14. Comparable increases in fibrinogen have been observed in previous studies in monkeys administered NEUMEGA. These increases were attributed to the transient acute phase reaction induced by IL-11.

A slight decrease in albumin (17% to 23%) was observed at day 7 in all animals administered NEUMEGA and a partial resolution occurred by day 14. This slight decrease is attributed to a transient acute phase response. Although a plasma volume expansion may have also contributed.

The results obtained in this Second Study have shown that resolution of CMC-544-related platelet count decreases occurred slightly earlier in monkeys given NEUMEGA for 5 days beginning 6 hours after the single CMC-544 dose, than in the majority of animals given CMC-544 alone. CMC-related increases in liver-associated serum enzymes, AST and ALT occurred with somewhat lower magnitude and incidence in animals administered CMC-544+NEUMEGA than animals administered CMC-544 alone. NEUMEGA-related changes (decrease in albumin, transient increase in fibrinogen) observed in the present study were consistent with those previously reported.

### Example 3

### Effect of Calicheamicin Conjugates on Platelet Levels in the Mouse

Experimental Design:

On Day 0, mice will be bled for baseline platelet values. Mice will then be dosed with vehicle, CMC-544 or other calicheamicin conjugates at 4 μg/mouse i.v. or i.p. Higher or lower doses may also be administered. Blood will be sampled 72 hours (Day 3), 96 hours (Day 4), and 168 hours (Day 8) post drug administration.

Procedures.

A 25 gauge needle will be inserted into the tail vein of the mouse and then withdrawn allowing for a drop of blood to seep out. A 5 μL sample of the blood will be collected for analysis. Blood will be sampled on Day 0, before drug administration, and on Days 3, 4, and 8, for a total of 4 collections of 5 μL each (total of 20 μL).

Dosing: CMC-544 or other conjugates of calicheamicin will be administered once either intraperitonealy or intravenously at a dose of 4 μg of calicheamicin DMH. The dose volume will be 200 μL for either route of administration.

Blood Collection: 5 μL of blood will be collected from the tail vein.

Pain or Distress: No pain or distress is anticipated but if it occurs, the attending veterinarian will be consulted.

Animal Toxicity or Ill Effects: No overt toxic effects of CMC-544 have been observed in mice when administered at a dose of 4 μg or less.

Animal Disposition:

The study will be terminated after the final blood collection time point on Day 8 post-drug administration. Mice will be euthanized by CO₂ inhalation.

### Example 4

### Study of CMC-544 in Patients with B-Cell Non-Hodgkin's Lymphoma (NHL)

CMC-544, is an antibody-targeted chemotherapy agent composed of a monoclonal antibody, which specifically targets the CD22 antigen, conjugated to calicheamicin, a potent cytotoxic antitumor antibiotic. Malignant cells of mature B-lymphocyte lineage express CD22; CMC-544 may be useful for treating lymphomas of B-cell origin.

Methods: A phase I, dose escalation trial of CMC-544 is ongoing in patients with relapsed or refractory B-cell NHL across 13 European and US sites. CMC-544 is administered intravenously every 3-4 weeks at doses of 0.4, 0.8, 1.34, 1.8, and 2.4 mg/m². Standard safety and pharmacokinetic data and preliminary efficacy data (assessed using the International Workshop to Standardize Response Criteria for NHL) are being collected.

Enrollment of patients with any type of B-cell NHL, except for Burkitt's and lymphoblastic lymphomas, is allowed in the dose escalation phase (1-6 patients per cohort). After the maximum tolerated dose (MTD) was identified, 15 patients with follicular lymphoma and 15 patients with diffuse large B-cell lymphoma will be enrolled in an expanded MTD cohort.

Results: As of June 2005, 34 patients (8 women, median 71 years old; 26 men, median 62 years old; number of previous treatments, median 4, range 2-11) were enrolled. Dose escalation was based on 1^{st} cycle safety evaluations of patient cohorts. Dose-limiting toxicities (DTLs) were reported for dose levels of 1.34 mg/m² (grade 4 thrombocytopenia, 2/11 patients), 1.8 mg/m² (bleeding requiring platelet transfusion, 1/6 patients), and 2.4 mg/m² (grade 4 thrombocytopenia, 1/6 patients; grade 4 neutropenia for 7 days, 1/6 patients).

Thus, the MTD, the dose level prior to the one where >33% DTLs occurred, was 1.8 mg/m². The most common drug-related adverse events (AEs, all grades) were: thrombocytopenia (65%), asthenia (47%), nausea (41%), neutropenia (29%), elevated liver function tests (27%), anorexia (14%), and epistaxis (12%). Grade 3-4 AEs that occurred with a frequency≧10% included: thrombocytopenia (38%), asthenia (12%), and neutropenia (12%). The nadir of the thrompocytopenia was 9±2 days and platelet counts recovered spontaneously. No major bleeding episodes were reported. At the 1.8 and 2.4 mg/m² dose levels, data suggested a dose-dependent component of platelet recovery to baseline levels and dose delays were required between successive doses. CMC-544 and total calicheamicin exposures in serum increased with dose. Clearance after 2^{nd} and 3^{rd} doses decreased approximately 8-14 fold compared with 1^{st} dose, and half-life increased from approximately 1 day to 4 days. Available data suggested an association of peak CMC-544 exposures and/or total calicheamicin levels with decreases in platelet counts. Accordingly, patients in the expanded MTD cohort receive 1.8 mg/m² CMC-544 every 4 weeks. Preliminary antitumor activity was observed in most cohorts. Complete and/or partial responses were observed in the 0.8 mg/m² (1/3 patients), 1.34 mg/m² (3/9 patients), 1.8 mg/m² (2/5 patients), and 2.4 mg/m² (2/5 patients) cohorts, and in the 1^{st} 6 patients in the expanded MTD cohort (4/6 patients).

### Example 5

### Comparison of the Effects of CMC-544 and Carboplatin on Platelets and Thrombopoietin in the Mouse

Vehicle (PBS), Carboplatin (125 mg/kg i.p.) or CMC-544 (200 μg/kg i.p) was each administered to nude mice on Day 0. Blood was collected as described above on Day 0, Day 4, Day 7, Day 11 and Day 13 for measurement of circulating platelets and circulating thrombopoietin (TPO) levels.

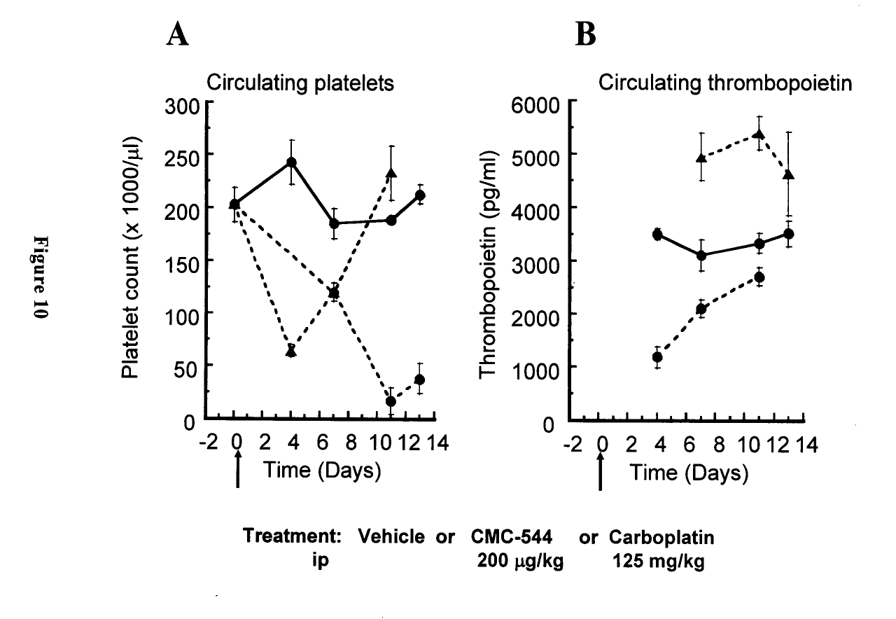

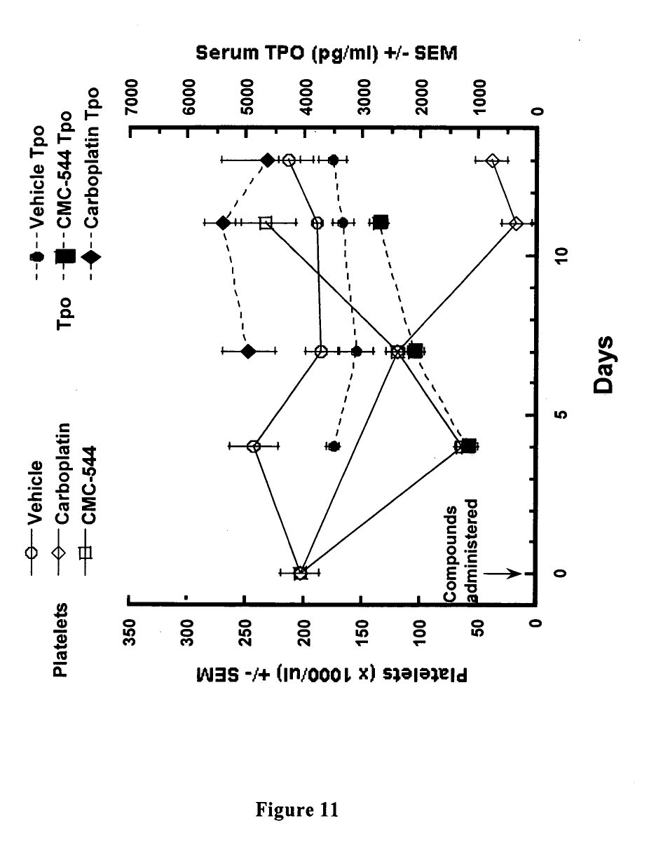

As shown on FIGS. 10 and 11, which present the results obtained in these experiments, CMC-544 was found to cause thrombocytopenia in mice with nadir on Day 3 or 4 after treatment; while Carboplatin causes thrombocytopenia with nadir on Day 10 or 11 after treatment.

Conventional chemotherapy is known to cause thrombocytopenia accompanied by increased levels of circulating thrombopoietin (TPO). In the present experiments, administration of Carboplatin was found to cause increased levels of circulating thrombopoietin (TPO) while a significant reduction in the levels of circulating TPO was observed in the case of CMC-544, suggesting that CMC-544 may inhibit the production of TPO. CMC-544-mediated inhibition of TPO production may be related to its effects on the liver function.

### Example 6

### Thrombocytopenia induced by CMC-544 and its Amelioration Using Oprelvekin (NEUMEGA®)

CMC-544 has demonstrated significant anti-tumor activity in pre-clinical and clinical trials in B-Non-Hodgkin's Lymphoma (B-NHL) patients (J. F. DiJoseph et al., Blood, 2004, 103: 1807-1814; J. F. DiJoseph et al., Clin. Cancer Res., 2004, 10: 8620-8629; J. F. DiJoseph et al., Cancer Immunol. Immunother., 2005, 54: 11-24; J. F. DiJoseph et al., Clin. Cancer Res., 2006, 12: 242-249; A Advani et al., Blood, 2005, 106: 230). In these trials, thrombocytopenia was reported as the most common dose-limiting adverse event. The present non-clinical study provides insights into the observed thrombocytopenia associated with CMC-544 treatment of B-NHL patients.

In vitro, CMC-544 neither bound to nor cause aggregation of platelets in platelet-rich plasma of human or murine origin. When administered to mice, a single dose of CMC-544 resulted in a 50% to 75% reduction in the number of circulating platelets with a nadir on day 3 or 4. The platelet values returned to normal at the latest by day 12. Similar effects were also observed in cynomolgus macaques with nadirs on day 4 or 5. Unconjugated anti-CD22 antibody, G5/44 (humanized IgG4 antibody) had no effect on circulating platelets. CMC-544-induced thrombocytopenia in mice was associated with a concomitant reduction in circulating levels of thrombopoietin (TPO) with a nadir on day 4. In contrast, treatment with a known cytotoxic chemotherapeutic agent, carboplatin, caused thrombocytopenia in mice but with a nadir on day 11. Unlike that with CMC-544, carboplatin-induced thrombocytopenia was associated with an increase in the levels of circulating TPO.

Subcutaneous pre-treatment of mice with oprelvekin (NEUMEGA®/rhIL-11) (250 μg/kg) significantly reduced the magnitude of the CMC-544-associated thrombocytopenia. When evaluated in cynomolgus macaques, daily subcutaneous pre-treatment with oprelvekin (125 μg/kg) for 5 days completely prevented CMC-544-associated thrombocytopenia but also suppressed elevations in serum hepatic aminotransferases associated with CMC-544 treatment alone. Concurrent administration of oprelvekin with CMC-544 failed to prevent the initial thrombocytopenia but did accelerate complete recovery of platelets counts to pre-treatment levels and also suppressed elevations in circulating serum hepatic aminotransferases.

This study further provides evidence that the prophylactic use of oprelvekin can ameliorate CMC-544-induced thrombocytopenia, potentially augmenting the therapeutic benefit of CMC-544 in patient with B-NHL.

Some of the results reported in this section have been presented at the American Society of Hematology annual meeting, Dec. 9-12, 2006, Orlando, Fla. in the following abstract: “Thrombocytopenia induced by CMC-544 and its amelioration using oprelvekin (NEUMEGA®/recombinant human interleukin-11”, by John F DiJoseph, Dana Walker, Maureen M Dougher, Doug C Armellino, Dave Clarke and Nitin K Damle.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method of alleviating thrombocytopenia in a subject, the method comprising a step of: administering a therapeutically effective amount of interleukin-11 to the subject, wherein thrombocytopenia is associated with administering to the subject a conjugate comprising a targeting moiety and a cytotoxic drug.

2. The method of claim 1, wherein the subject is suffering from cancer or a cancerous condition.

3. The method of claim 1 or 2, wherein the targeting moiety comprises an antibody.

4. The method of claim 3, wherein the antibody is an anti-CD22 antibody, an anti-CD33 antibody, an anti-Lewis Y antibody, an anti-5T4 antibody, an anti-CD30 antibody, or any combinations thereof.

5. The method of claim 1 or 2, wherein the cytotoxic drug is a calicheamicin, a calicheamicin derivative, an esperamicin, or an esperamicin derivative.

6. The method of claim 1 or 2, wherein the conjugate is an anti-CD22 antibody-calicheamicin conjugate.

7. The method of claim 1 or 2, wherein the conjugate is an anti-CD33 antibody-calicheamicin conjugate.

8. The method of claim 1 or 2, wherein the conjugate is an anti-Lewis Y antibody-calicheamicin conjugate.

9. The method of claim 1 or 2, wherein the conjugate is an anti-5T4 antibody-calicheamicin conjugate.

10. The method of claim 1 or 2, wherein the conjugate is an anti-CD30 antibody-calicheamicin conjugate.

11. The method of claim 1 or 2, wherein interleukin-11 comprises recombinant human interleukin-11.

12. The method of claim 1 or 2, wherein interleukin-11 is administered prior to administration of the conjugate.

13. The method of claim 1 or 2, wherein said method prevents, reduces, slows down or stops thrombocytopenia in the subject.

14. The method of claim 1 or 2, wherein thrombocytopenia is at least partly resulting from bone marrow destruction.

15. The method of claim 14, wherein the targeting moiety comprises an antibody.

16. The method of claim 15, wherein the antibody is an anti-CD22 antibody, an anti-CD33 antibody, an anti-Lewis Y antibody, an anti-5T4 antibody, an anti-CD30 antibody, or any combinations thereof.

17. The method of claim 14, wherein the cytotoxic drug is a calicheamicin, a calicheamicin derivative, an esperamicin, or an esperamicin derivative.

18. The method of claim 14, wherein the conjugate is an anti-CD22 antibody-calicheamicin conjugate.

21. The method of claim 14, wherein the conjugate is an anti-5T4 antibody-calicheamicin conjugate.

22. The method of claim 14 wherein the conjugate is an anti-CD30 antibody-calicheamicin conjugate.

23. The method of claim 14, wherein interleukin-11 comprises recombinant human interleukin-11.

24. The method of claim 14, wherein interleukin-11 is administered prior to administration of the conjugate.

25. The method of claim 14, wherein said method prevents, reduces, slows down or stops thrombocytopenia at least partly resulting from bone marrow destruction.

26. The method of claim 1 or 2, wherein thrombocytopenia is at least partly resulting from liver damage.

27. The method of claim 26, wherein the targeting moiety comprises an antibody.

28. The method of claim 27, wherein the antibody is an anti-CD22 antibody, an anti-CD33 antibody, an anti-Lewis Y antibody, an anti-5T4 antibody, an anti-CD30 antibody, or any combinations thereof.

29. The method of claim 26, wherein the cytotoxic drug is a calicheamicin, a calicheamicin derivative, an esperamicin, or an esperamicin derivative.

30. The method of claim 26, wherein the conjugate is an anti-CD22 antibody-calicheamicin conjugate.

31. The method of claim 26, wherein the conjugate is an anti-CD33 antibody-calicheamicin conjugate.

32. The method of claim 26, wherein the conjugate is an anti-Lewis Y antibody calicheamicin conjugate.

33. The method of claim 26, wherein the conjugate is an anti-5T4 antibody-calicheamicin conjugate.

34. The method of claim 26 wherein the conjugate is an anti-CD30 antibody-calicheamicin conjugate.

35. The method of claim 26, wherein interleukin-11 comprises recombinant human interleukin-11.

36. The method of claim 26, wherein interleukin-11 is administered prior to administration of the conjugate.

37. The method of claim 26, wherein said method prevents, reduces, slows down or stops thrombocytopenia at least partly resulting from liver damage.

38. The method of claim 37, wherein said method further prevents, reduces, slows down or stops liver damage in the subject.

39. The method of claim 37, wherein said method further prevents, reduces, slows down or stops liver-damage related inflammation in the subject.

40. A pharmaceutical composition comprising a therapeutically effective amount of interleukin-11, at least one conjugate whose administration results in thrombocytopenia, and at least one physiologically acceptable carrier, wherein the conjugate comprises a targeting moiety and a cytotoxic drug.

41. The pharmaceutical composition of claim 40, wherein the targeting moiety comprises an antibody.

42. The pharmaceutical composition of claim 41, wherein the antibody is an anti-CD22 antibody, an anti-CD33 antibody, an anti-Lewis antibody, an anti-5T4 antibody, an anti-CD30 antibody, or any combinations thereof.

43. The pharmaceutical composition of claim 40, wherein the cytotoxic drug is a calicheamicin, a calicheamicin derivative, an esperamicin, or an esperamicin derivative.

44. The pharmaceutical composition of claim 40, wherein the conjugate is an anti-CD22-antibody-calicheamicin conjugate.

45. The pharmaceutical composition of claim 40, wherein the conjugate is an anti-CD33-antibody-calicheamicin conjugate.

46. The pharmaceutical composition of claim 40, wherein the conjugate is an anti-Lewis Y antibody-calicheamicin conjugate.

47. The pharmaceutical composition of claim 40, wherein the conjugate is an anti-5T4 antibody-calecheamicin conjugate.

48. The method of claim 26 wherein the conjugate is an anti-CD30 antibody-calicheamicin conjugate.

49. The pharmaceutical composition of claim 40, wherein interleukin-11 comprises recombinant human interleukin-11.

50. The pharmaceutical composition of claim 40 or 49, wherein interleukin-11, the at least one conjugate, and physiologically acceptable carrier are combined as one or more preparations for simultaneous or sequential administration of interleukin-11 and the at least one conjugate.

51. The pharmaceutical composition of claim 40, wherein administration of said composition to a subject prevents, reduces or stops thrombocytopenia in the subject.

52. The pharmaceutical composition of claim 51, wherein the subject suffers from cancer or a cancerous condition.

53. The pharmaceutical composition of claim 40, wherein thrombocytopenia produced by administration of the at least one conjugate results, at least partly, from bone marrow destruction.

54. The pharmaceutical composition of claim 40, wherein thrombocytopenia produced by administration of the at least one conjugate results, at least partly, from liver damage.

55. A kit comprising:
interleukin-11; and
at least one conjugate whose administration to a subject results in thrombocytopenia in the subject, wherein the conjugate comprises a targeting moiety and a cytotoxic drug.

56. The kit of claim 55, wherein the targeting moiety comprises an antibody.

57. The kit of claim 56, wherein the antibody is an anti-CD22 antibody, an anti-CD33 antibody, an anti-Lewis antibody, an anti-5T4 antibody, an anti-CD30 antibody, or any combinations thereof.

58. The kit of claim 55, wherein the cytotoxic drug is a calicheamicin, a calicheamicin derivative, an esperamicin, or an esperamicin derivative.

59. The kit of claim 55, wherein the conjugate is an anti-CD22-antibody-calicheamicin conjugate.

62. The kit of claim 55, wherein the conjugate is an anti-5T4-antibody-calicheamicin conjugate.

63. The method of claim 55 wherein the conjugate is an anti-CD30 antibody-calicheamicin conjugate.

64. The kit of claim 55, wherein interleukin-11 comprises recombinant human interleukin-11.

65. The kit of claim 55, wherein thrombocytopenia produced by the at least one conjugate results, at least partly, from bone marrow destruction.

66. The kit of claim 55, wherein thrombocytopenia produced by the at least one conjugate results, at least partly, from liver damage.
